(19) United States
(12) Patent Application Publication
(10) Pub. No.: US 2022/0347353 A1
(43) Pub. Date: Nov. 3, 2022

(54) METHODS FOR TREATING ACUTE WOUNDS AND IMPROVING OUTCOMES

(71) Applicant: Stratatech Corporation, Madison, WI (US)

(72) Inventors: Janice Smiell, Madison, WI (US); Allen Comer, M, WI (US); Mary Lokuta, Madison, WI (US); B. Lynn Allen-Hoffmann, Madison, WI (US)

(21) Appl. No.: 17/763,552

(22) PCT Filed: Sep. 28, 2020
(86) PCT No.: PCT/US2020/053061

§ 371 Date: Mar. 24, 2022

Related U.S. Application Data
(60) Provisional application No. 62/907,308, filed on Sep. 27, 2019.
(60) Provisional application No. 62/910,887, filed on Oct. 4, 2019.
(60) Provisional application No. 62/979,649, filed on Feb. 21, 2020.

(51) Int. Cl. A61L 27/60; A61L 27/38; A61L 27/24

(52) U.S. Cl. CPC A61L 27/60; A61L 27/3813; A61L 27/24

(57) ABSTRACT

The present disclosure provides methods for treating acute wounds and improving outcomes by applying to an acute wound a skin substitute that is an organotypic human skin equivalent comprising NIKS cells. In certain embodiments, the closed wound has improved vascularity, improved pigmentation, decreased thickness, decreased pain, increased pliability, increased surface area, decreased stiffness, decreased itching, improved color, or any combination thereof, as assessed by an observer or by the subject, as compared to an autograft or another skin substitute.

### CROSS REFERENCE TO RELATED APPLICATIONS

This application claims priority to U.S. Provisional Application No. 62/907,308, filed Sep. 27, 2019, U.S. Provisional Application No. 62/910,887, filed Oct. 4, 2019, and U.S. Provisional Application No. 62/979,649, filed Feb. 21, 2020, the disclosures of which are incorporated herein by reference.

### FIELD OF THE INVENTION

The present invention encompasses methods for treating acute wounds and improving outcomes by applying to an acute wound a skin substitute.

### BACKGROUND OF THE INVENTION

Effective treatment of acute wounds often requires skin grafting. Autografts or autologous skin grafts, the standard of care, utilize surgical harvesting of healthy skin from the patient for subsequent transplantation to the acute wound following excision of nonviable tissue. This procedure results in an iatrogenic donor-site wound that receives medical management of pain and is associated with scarring, itching, chronic impairment of skin function, infection, and the like. Reducing or eliminating the surgical harvesting of skin for autografting may minimize acute pain, reduce and improve quality of life for the patient.

Certain patient populations have an even greater need for an alternative to autografting. For instance, young children have limited surface area available as donor sites. Similarly, patients with acute wounds covering a high total body surface area also have limited donor sites. And in elderly patients, harvest of donor sites can be contraindicated to avoid the creation of a full-thickness (FT) wound due to thinning dermis. Underlying comorbidities can also contribute to slow wound healing in a patient, particularly in elderly patients. It is clear therefore that patients and providers need alternative approaches that provide immediate wound coverage and promote definitive wound closure, while reducing and/or eliminating the need for autograft harvest.

Allografts, which use skin from another human (e.g., cadaver, other donor source, etc.), and xenografts, which use skin from another species (e.g., porcine or bovine grafts), do not fulfill the need, as they are usually only temporary skin replacements.

Similarly, while advances in tissue engineering have led to the development of a wide range of skin substitutes for wound healing, most skin substitutes do not claim to promote wound closure without the need for subsequent autografting. For instance, according to a 2018 article in Int J Burns Trauma (2018: 8(4): 77-87), Integra® artificial skin is the most widely accepted artificial skin substitute for management of acute deep partial-thickness and full-thickness burns. This skin substitute is a bilayer consisting of a temporary epidermal substitute layer of silicone and a dermal replacement layer consisting of cross-linked bovine tendon collagen and glycosaminoglycan. Yet, “successful treatment” means that after approximately 14-30 days there is adequate vascularization of the dermal layer, the temporary silicone layer is removed, and then a thin split-skin autograft is placed over the vascular “neodermis”. Even then, a randomized trial found that the median artificial skin substitute take was 80% compares with 95% for all comparative sites (e.g., autograft, allograft, xenograft, or a synthetic dressing). See, Heimback et al., Ann. Surg, 1988, 208(3): 313-319. It is also reported that meticulous surgical technique and appropriate postoperative care are critical for a successful outcome. See, for instance, Sterling et al., Management of the burn wound. In: ACS surgery: principles and practice. Hamilton, ON: Decker; 2010. http://dx.doi.org/10.2310/7800.S07C15.

Accordingly, there remains a need in the art for improved treatment methods that eliminate or reduce the need for autografting.

### SUMMARY OF THE INVENTION

In an aspect, the present disclosure encompasses a method for closing an acute wound in a subject, the method comprising applying a skin substitute over an acute wound and allowing the wound to heal, wherein the skin substitute is an organotypic human skin equivalent comprising NIKS. In certain embodiments, the closed wound has improved vascularity, improved pigmentation, decreased thickness, decreased pain, increased pliability, increased surface area, decreased stiffness, decreased itching, improved color, or any combination thereof, as assessed by an observer or by the subject, as compared to an autograft or another skin substitute.

In another aspect, the present disclosure encompasses a method for closing an acute wound in a subject, the method comprising applying a skin substitute over an acute wound that contains intact dermal elements and allowing the wound to heal, wherein the skin substitute is an organotypic human skin equivalent comprising NIKS. In certain embodiments, the closed wound has improved vascularity, improved pigmentation, decreased thickness, decreased pain, increased pliability, increased surface area, decreased stiffness, decreased itching, improved color, or any combination thereof, as assessed by an observer or by the subject, as compared to an autograft or another skin substitute.

In another aspect, the present disclosure encompasses a method for improving an outcome of skin grafting in a subject, the method comprising applying a skin substitute over an acute wound, wherein the skin substitute is an organotypic human skin equivalent comprising NIKS, and wherein the outcome is reduced autografting, improved vascularity, improved pigmentation, decreased thickness, decreased pain, increased pliability, increased surface area, decreased stiffness, decreased itching, improved color, decreased infection rate or any combination thereof, as assessed by an observer or by the subject, as compared to an autograft.

In another aspect, the present disclosure encompasses a method for improving an outcome of skin grafting in a subject, the method comprising applying a skin substitute over an acute wound that contains intact dermal elements, wherein the skin substitute is an organotypic human skin equivalent comprising NIKS, and wherein the outcome is reduced autografting, improved vascularity, improved pigmentation, decreased thickness, decreased pain, increased pliability, increased surface area, decreased stiffness, decreased itching, improved color, decreased infection rate or any combination thereof, as assessed by an observer or by the subject, as compared to an autograft.

In all the above aspects, wound closure or the improved outcome can occur without the application of any autologous tissue or any autologous cells over or under the skin substitute. In certain embodiments, wound closure or the improved outcome may occur without the application of any autologous tissue or any autologous cells.

Other aspects and iterations of the invention are described more thoroughly below.

### BRIEF DESCRIPTION OF THE FIGURES

The application file contains at least one photograph executed in color. Copies of this patent application publication with color photographs will be provided by the Office upon request and payment of the necessary fee.

FIG. 1A is a graph depicting percent treatment area autografted at day 28 for cohorts 1-3. Data is from the intent-to-treat population.

FIG. 1B is a graph depicting percent of subjects with wound closure by 3 months for cohorts 1-3. Data is from the intent-to-treat population. One subject in cohort 3 was lost to follow-up by month 3. Wound closure is defined for this figure as 95% re-epithelialization with absence of drainage.

FIG. 2A is a graph depicting percent mean estimated re-epithelialization for StrataGraft treatment sites (y-axis) at various time points (x-axis) for cohorts 1 (circle), 2 (triangle), and 3 (square). Data is from the intent-to-treat population. One subject in cohort 1 was lost to follow-up after month 3; one subject in cohort 3 missed the study session on day 28 and was lost to follow-up by month 3 and a second subject in cohort 3 was lost to follow-up after month 3. An additional subject in cohort 3 was missing at month 6, but returned at month 12, so did not discontinue from the study.

FIG. 2B is a graph depicting percent mean estimated re-epithelialization for autograft treatment sites (y-axis) at various time points (x-axis) for cohorts 1 (circle), 2 (triangle), and 3 (square). Data is from the intent-to-treat population. One subject in cohort 1 was lost to follow-up after month 3; one subject in cohort 3 missed the study session on day 28 and was lost to follow-up by month 3 and a second subject in cohort 3 was lost to follow-up after month 3. An additional subject in cohort 3 was missing at month 6, but returned at month 12, so did not discontinue from the study.

FIG. 3A is a graph depicting POSAS score as determined by a trained observer. Data are from the intent-to-treat population. Observer assessment scale per parameter of total score: 1=normal skin; 10=worst scar imaginable. The total score is the summation of the values for each of the 6 questions within the assessor. Horizontal dashed line indicates where the lowest possible (best possible) score falls. SD=standard deviation.

FIG. 3B is a graph depicting POSAS score as determined by the subject. Data are from the intent-to-treat population. Subject assessment scale per parameter of total score: 1=no, not at all; 10=yes, very much (pain and itching assessment) or yes, very different (color, stiffness, thickness, and irregularity assessments). The total score is the summation of the values for each of the 6 questions within the assessor. Horizontal dashed line indicates where the lowest possible (best possible) score falls. SD=standard deviation.

FIG. 4A is a graph depicting the mean percent treatment area that was autografted at day 28 (top) or month 3 (bottom), by size of treatment area, for the autografted treatment site (grey, top bar) and the StrataGraft treatment site (blue, bottom bar).

FIG. 4B is a graph depicting the percent wound closure at month 3 (bottom), by size of treatment area, for the autografted treatment site (grey, top bar) and the StrataGraft treatment site (blue, bottom bar).

FIG. 5 is a schematic depicting the patient population for the Phase 3 trial. ITT=intention-to-treat population. *Wound excluded because surgeon deemed autografting was not necessary. **Patients were discontinued from the study prior to database lock for the coprimary endpoints.

FIG. 6A is a graph showing the mean percentage area of StrataGraft and autograft treatment sites that were autografted by Month 3 in the modified intention-to-treat population (mITT). ^{a}P value from one-sided Wilcoxon Signed Rank Test. ^{b}Percentage area autografted by Month 3 is the sum of the percentage areas at each visit, up to and including the Month 3 visit. CI=Confidence Interval. SD=Standard Deviation.

FIG. 6B is a graph showing the mean percentage area of treatment sites requiring autograft over time in the modified intention-to-treat population (mITT).

FIG. 7A is a graph showing the percentage of patients that achieved durable wound closure of the StrataGraft and autograft treatment sites by Month 3 in the modified intention-to-treat population (mITT). CI=Confidence Interval.

FIG. 7B is a graph showing the proportion of patients achieving durable wound closure without regraft at an autograft site, or autograft at a StrataGraft site in the modified intention-to-treat population (mITT).

FIG. 8 is a graph showing Patient and Observer Scar Assessment Scale (POSAS) observer scores for the donor site at Month 3 in the modified intention-to-treat population (mITT). In each category, a lower POSAS score is more favorable of cosmesis.

FIG. 9A are photographic images of StrataGraft and autograft treatment sites from a representative patient. The patient sustained a 17% TBSA thermal burn to the head, neck, bilateral upper and lower extremities and was treated with 680 cm² StrataGraft tissue. By Day 28, the StrataGraft treatment site was 95% reepithelialized and was completely closed at Month 2

FIG. 9B are photographic images of StrataGraft and autograft donor sites from a representative patient. The patient is the same as in FIG. 9A.

FIG. 10A are photographic images of StrataGraft and autograft treatment sites from a representative patient. The patient sustained a 14% TBSA thermal burn to bilateral upper extremities, face, and bilateral feet. The right proximal forearm was treated with 135 cm² StrataGraft tissue. By Day 28, the StrataGraft treatment site was 95% reepithelialized and was completely closed at Month 2

FIG. 10B are photographic images of StrataGraft and autograft donor sites from a representative patient. The patient is the same as in FIG. 10A.

### DETAILED DESCRIPTION

In one aspect, the present disclosure provides methods for closing a variety of acute wounds. As used herein, the term “wound closure” means ≥95% re-epithelialization of the wound surface with absence of drainage. Methods of the present disclosure for closing acute wounds provide improved outcomes for the subject compared to the current standard-of-care, surgical excision and autografting, (e.g., an off-the-shelf product, a primary intervention without the need for autografting, reduced autografting, effectiveness in vulnerable patient populations, improved outcomes, etc.).

In another aspect, the present disclosure provides methods for improving an outcome of skin grafting, particularly in vulnerable patient populations. Non-limiting examples of improved outcomes include reduced autografting, improved pigmentation or coloring at a graft site and/or a donor site, decreased scar thickness at a graft site, decreased pain, increased pliability at a graft site, decreased stiffness at a graft site, decreased itching at a graft site, decreased infection rate, decreased donor-site morbidities, and any combination thereof, as assessed by an observer or by the subject, as compared to an autograft only. Donor-site morbidities include but are not limited to tenderness, pain, cold-sensitivity, scarring in general or more particularly hypertrophic scarring, infection, conversion from a split-thickness wound to a complex wound or a full thickness wound, etc.

Methods of the present disclosure comprise applying a skin substitute over an acute wound, wherein the skin substitute is an organotypic human skin equivalent comprising NIKS cells. The skin substitute may be applied to the wound within hours or days of the wound's formation, within the first or second week of the wound's formation, or even later. Wounds are typically prepared for treatment by methods known in the art, for instance cleansing with soap and water, surgical excision (fascial excision or tangential excision) if clinically indicated and feasible, etc. The location of the wound is not limiting. For instance, a wound may be on an arm, a leg, a torso, a face, a head, a neck, a hand, a foot, a buttock, over joints, etc. In some embodiments, the method consists of a single application of the skin substitute. In other embodiments, the method comprises 2 or more applications of the skin substitute.

Other aspects of the disclosure are described in further detail below.

### I. Skin Substitute

Methods of the present disclosure require application of a skin substitute that is an organotypic human skin equivalent comprising NIKS® cells. Accordingly, the skin substitute is an engineered (non-natural), bilayer tissue designed to mimic natural human skin with both an inner dermis-like layer and an outer epidermis-like layer. The viable cells of the skin substitute are metabolically active and secrete a spectrum of growth factors, chemotactic factors, cytokines, inflammatory mediators, enzymes, and host defense peptides that condition the wound bed, promote tissue regeneration and repair, and reduce infection. Production of the skin substitute by organotypic culture produces a well-developed epidermal layer of fully-stratified human keratinocytes that exhibits barrier function comparable to that of intact human skin.

The inner dermis-like layer is also referred to as a “dermal equivalent.” Suitable dermal equivalents are a matrix comprising gelled collagen and human dermal fibroblasts. In exemplary embodiments, the human dermal fibroblasts are primary normal human dermal fibroblasts. The collagen present in the dermal equivalent may include type I rat tail collagen. Alternatively, the only collagen present in the dermal equivalent may be produced by cells of the skin substitute (e.g., human dermal fibroblasts). The matrix may further comprise additional biomolecules produced by the cells contained therein. In an exemplary embodiment, the dermal layer is composed of normal human dermal fibroblasts embedded within an extracellular matrix produced and organized by the fibroblasts. For the avoidance of doubt, in this embodiment, there is no non-human collagen in the dermal layer. In another exemplary embodiment, the dermal layer is composed of normal human dermal fibroblasts embedded in a gelled-collagen matrix that contains purified rat-tail tendon type I collagen. For the avoidance of doubt, in this embodiment, although the rat-tail tendon type I collagen is gelled to give the dermal layer its primary structure, the normal human dermal fibroblasts embedded therein may produce and contribute collagen (and other biomolecules) to the matrix.

The outer epidermis-like layer comprises NIKS® cells. NIKS® cells were deposited with the ATCC (CRL-12191) and are described in further detail in U.S. Pat. Nos. 5,989,837 and 6,964,869, the disclosures of which are incorporated herein by reference. The phrase “an organotypic human skin equivalent comprising NIKS cells” encompasses NIKS® cells engineered to express a variety of exogenous nucleic acids that may provide a beneficial effect in wound closing (e.g., encode a protein that directly or indirectly promotes wound healing). Expressly contemplated are NIKS® cells engineered to express an exogenous gene encoding a VEGF protein (e.g., VEGF-A, etc.), an exogenous gene encoding a hypoxia-inducible factor (e.g., HIF-1A, etc.), an exogenous gene encoding an angiopoietin (e.g., ANGPT1, etc.), an exogenous gene encoding a cathelicidin peptide or a cleavage product thereof (e.g., hCAP-18, etc.), an exogenous gene encoding a beta-defensin (e.g., hBD-3, etc.), an exogenous gene encoding a keratinocyte growth factor (e.g., KGF-2, etc.), an exogenous gene encoding a tissue inhibitor of metalloproteinases (e.g., TIMP-1, etc.), an exogenous IL-12 gene, as well exogenous nucleic acid sequences encoding other antimicrobials, growth factors, transcription factors, interleukins and extracellular matrix proteins. As non-limiting examples, see for instance, U.S. Pat. Nos. 7,498,167, 7,915,042, 7,807,148, 7,988,959, 8,808,685, 7,674,291, 8,092,531, 8,790,636, 9,526,748, 9,216,202, and 9,163,076, and US 20190030130, the disclosures of which are incorporated herein by reference.

Suitable manufacturing processes for producing a skin substitute that is an organotypic human skin equivalent comprising NIKS® cells have been previously described in the art. See, for instance, U.S. Pat. Nos. 7,498,167, 7,915,042, 7,807,148, 7,988,959, 8,808,685, 7,674,291, 8,092,531, 8,790,636, 9,526,748, 9,216,202, 9,163,076 and 10,091,983, and US 20190030130, the disclosure of which are incorporated by reference in their entirety. Advantageously, skin substitutes produced as described above have excellent handling characteristics that enable them to be meshed, sutured, stapled or secured with an adhesive, in the same manner as autologous skin grafts, cadaver allografts and xenografts.

In an exemplary embodiment, the skin substitute is StrataGraft™. In another exemplary embodiment, the skin substitute is an ExpressGraft™ tissue.

### II. Acute Wound

The terms “skin wound” and “wound” are used interchangeably herein, and refer to a breach in continuity of skin. Skin wounds are defined in the art as acute or chronic. An acute wound has normal wound physiology, and healing is anticipated to progress through the normal stages of wound healing. A chronic wound is defined as one that has failed to pass through the normal healing process—i.e., does not heal in an orderly set of stages and in a predictable amount of time (e.g., a three to four week period of time).

Acute wounds are generally classified based on the mode of infliction and/or causative agent. Methods of the present disclosure may be used to treat a variety of acute wound types including, but not limited to, surgical wounds, burn wounds, bite wounds, puncture wounds, sharp cuts, lacerations, etc. In exemplary embodiments, methods of the present disclosure may be used to treat a surgical wound. Non-limiting examples of surgical wounds that may be treated include donor sites for grafts, post-laser surgery wounds, post-podiatric procedure wounds, cosmetic surgery wounds, cancer excisions, wounds generated by the treatment of scar contractures, etc. In other exemplary embodiments, methods of the present disclosure may be used to treat a burn wound. In some examples, the wound is from a thermal burn. A “thermal burn” refers to a burn cause by fire, hot objects, steam, or hot liquids. In some examples, the wound is from an electrical burn. An “electrical burn” refers to a burn caused by contact with electrical sources, including a lightning strike. In some examples, the wound is from a radiation burn. A “radiation burn” refers to a burn caused by prolonged exposure to sources of UV radiation such as sunlight (e.g., sunburn), tanning booths, or sunlamps or by X-rays, radiation therapy or radioactive fallout. In some examples, the wound is from a chemical burn. A “chemical burn” refers to a burn caused by contact with highly acidic or basic substances. In some examples, the wound is from a friction burn. A “friction burn” refers to a burn caused by friction between the skin and hard surfaces, such as roads, carpets, floors, etc.

In each of the above embodiments, the acute wound may be a full-thickness wound, a partial-thickness wound, or a complex wound that contains intact dermal elements. In certain embodiments, the wound is a partial-thickness wound or a complex wound that contains intact dermal elements. The term “full-thickness” in the context of a skin wound is defined as penetrating the epidermis and dermis but not extending beyond the subcutaneous tissue. The term “partial-thickness” in the context of a skin wound is defined as penetrating the epidermis and extending into, but not penetrating, the dermis. The dermis itself is divided into two regions, the uppermost being the papillary region. This area is composed mostly of connective tissue and serves only to strengthen the connection between the epidermis and the dermis. Partial-thickness wounds that only extend down to this layer of the skin are considered superficial partial thickness wounds. The reticular region of the dermis contains not only connective tissue, but hair follicles, sebaceous and sweat glands, cutaneous sensory receptors, and blood vessels. Damage to this layer of the skin is classified as a deep partial-thickness wound, and can lead to significant scarring. The term “complex wound” in the context of a skin wound means the wound has different depths or etiologies across it. The term “containing intact dermal elements” means there are portions of skin structures (e.g., hair follicles, sweat glands, etc.) remaining in the bed of the wound that can provide a source of keratinocytes or keratinocyte precursor cells for migration to the surface of the wound bed. The terms “partial-thickness” and “full-thickness” are mutually exclusive but may coincide across a complex wound. For instance, a “complex wound that contains intact dermal elements” may refer to a wound that has both full-thickness characteristics (e.g., no intact dermal elements, etc.) and partial-thickness characteristics (e.g., intact dermal elements, etc.).

In the management of burn wounds, it is known that an acute burn wound may continue to progress over the first few days leading to a change in classification of the wound. As a non-limiting example, a superficial burn wound may progress to a partial-thickness burn wound, a complex burn wound that contains intact dermal elements, or a full-thickness burn wound over a period of days. Alternatively, or in addition to change in the depth of the burn, an increase in burn surface area may occur. This phenomenon is referred to as “burn wound conversion,” and the wound is then referred to as “a secondary burn wound.” This progression is not considered a failure to pass through the normal healing process. As such, in some embodiments, an acute wound may be a secondary burn wound.

Burn wounds have also been classified by the degree of the burn. A third degree burn is a full thickness burn wound and a second degree burn is a partial thickness burn wound. However, many burn wounds are complex wounds, with different depths across the entirety of the wound. In clinical practice, these wounds are often classified based on the most severe aspect of the wound when using the degree classification system.

Methods of the present disclosure may be used to treat acute wounds that vary in size, and an advantage of the methods of the present disclosure is that there are no limits on the wound size.

In the above embodiments, an acute wound may be less than 200 cm². For instance, the wound may be less than 150 cm², less than 100 cm², less than 50 cm², less than 10 cm², or less than 1 cm². In some embodiments, the wound may be about 1 cm² to 199 cm². For instance, the wound may be 0.5 cm² to about 195 cm², 0.5 cm² to about 150 cm², 0.5 cm² to about 100 cm², or 0.5 cm² to about 50 cm². Alternatively, the wound may be about 5 cm² to about 195 cm², about 5 cm² to about 150 cm², about 5 cm² to about 100 cm², or about 5 cm² to about 50 cm². Alternatively, the wound may be about 50 cm² to about 195 cm², about 50 cm² to about 150 cm², or about 50 cm² to about 100 cm².

Alternatively, in the above embodiments, an acute wound may be greater than or equal to 200 cm². For instance, the wound may be greater than or equal to 250 cm², greater than or equal to 300 cm², greater than or equal to 350 cm², or greater than or equal to 400 cm². The wound may also be greater than or equal to 500 cm², greater than or equal to 600 cm², greater than or equal to 700 cm², greater than or equal to 800 cm², greater than or equal to 900 cm², or greater than or equal to 1000 cm². The wound may also be greater than or equal to 2000 cm², greater than or equal to 3000 cm², greater than or equal to 4000 cm², greater than or equal to 5000 cm², or greater than or equal to 10,000 cm². In some embodiments, the wound may be 200 cm² to 1000 cm². For instance, the wound may be about 200 cm² to about 950 cm², about 200 cm² to about 900 cm², about 200 cm² to about 800 cm², about 200 cm² to about 700 cm², or about 200 cm² to about 600 cm². Alternatively, the wound may be about 200 cm² to about 500 cm², about 200 cm² to about 450 cm², or about 200 cm² to about 400 cm². In some embodiments, the wound may be 200 cm² to 2000 cm². For instance, the wound may be about 200 cm² to about 1950 cm², about 200 cm² to about 1900 cm² about 200 cm² to about 1800 cm², about 200 cm² to about 1700 cm², or about 200 cm² to about 1600 cm². Alternatively, the wound may be about 200 cm² to about 1500 cm², about 200 cm² to about 1450 cm², or about 200 cm² to about 1400 cm². Alternatively, the wound may be about 1000 cm² to about 1950 cm², about 1000 cm² to about 1900 cm², about 1000 cm² to about 1800 cm², about 1000 cm² to about 1700 cm², or about 1000 cm² to about 1600 cm². Alternatively, the wound may be about 1000 cm² to about 1500 cm², about 1000 cm² to about 1450 cm², or about 1000 cm² to about 1400 cm². Alternatively, the wound may be about 1000 cm² to about 15,000 cm², about 3500 cm² to about 15,000 cm², or about 5000 cm² to about 15,000 cm².

Methods of the present disclosure may be used to treat uninfected acute wounds or wounds that are deemed clinically infected. In embodiments where the wound is deemed clinically infected, it may be advantageous to use an organotypic human skin equivalent comprising NIKS® cells engineered to express one or more antimicrobial peptide.

Methods of the present disclosure may be particularly preferred for acute wounds where surgical intervention (e.g., excision and split thickness autografting) is clinically indicated.

### III. Subject

A “subject,” as used herein, refers to a human with an acute wound, as the term is described in Section II. In some embodiments, the subject has an acute wound where surgical intervention is clinically indicated (e.g., debridement and split-thickness autografting). In further embodiments, the subject has a partial-thickness wound or a complex wound that contains intact dermal elements. In still further embodiments, the acute would is from a burn or is a surgical wound. In still further embodiments, the acute would is from a thermal burn.

In some embodiments, a subject is 18 to 64 years in age. Surprisingly, applicants have discovered that methods of the present disclosure also effectively treat acute wounds in subjects less than 18 years of age and in subjects 65 years and older.

In each of the above embodiments, the subject's total body surface area covered by the acute wound or more likely by a plurality of acute wounds, referred to herein as “% TBSA,” may vary. In some embodiments, the % TBSA is at least 1%, at least 2%, or at least 5%. In some embodiments, the % TBSA is greater than 5%, greater than 10%, greater than 15%, or greater than 20%. In some embodiments, the % TBSA is greater than 25%. In some embodiments, the % TBSA is about 1% to about 90%, or about 1% to about 85%. In some embodiments, the % TBSA is about 1% to about 60%. In some embodiments, the % TBSA is about 1% to about 50%. In some embodiments, the % TBSA is about 5% to about 50%. In some embodiments, the % TBSA is about 10% to about 50%. In some embodiments, the % TBSA is about 15% to about 50%. In some embodiments, the % TBSA is about 20% to about 50%. In some embodiments, the % TBSA is about 25% to about 50%. In some embodiments, the % TBSA is about 10% to about 90%. In some embodiments, the % TBSA is about 15% to about 90%. In some embodiments, the % TBSA is about 20% to about 90%. In some embodiments, the % TBSA is about 25% to about 90%. In some embodiments, the % TBSA is about 10% to about 85%. In some embodiments, the % TBSA is about 15% to about 85%. In some embodiments, the % TBSA is about 20% to about 85%. In some embodiments, the % TBSA is about 25% to about 85%.

Methods of the present disclosure may be particularly preferred for vulnerable subjects including but not limited to subjects with limited surface area available for donor sites, subjects for whom harvest of donor sites is contraindicated, subjects with a high Baux score, subjects that are not hemodynamically stable, subjects that are at risk for delayed or impaired wound healing, or any combination thereof. Non-limiting examples of vulnerable subjects with limited surface area available for donor sites include subjects with a high % TBSA, such as a % TBSA of about 25% or greater, about 30% or greater, or about 35% or greater; subjects 10 years of age or younger, 5 years of age or younger, or 1 year of age or younger; and subjects 65 years of age or older, 70 years of age or older, 75 years of age or older, or 80 years of age or older. A vulnerable subject with a high Baux score typically has a Baux score of 100 or more. Non-limiting examples of subjects at risk for delayed or impaired wound healing include subjects with thinning dermis, inhalation injury or other coexisting injuries, a ≥25% TBSA, diabetes, infection (typically at the wound site but may be elsewhere), obesity, medications known to impair wound healing, a smoking habit, excessive alcohol drinking, low blood pressure, vascular disease, edema, cancer, malnutrition, or any combination thereof. A vulnerable subject with thinning dermis typically has a dermal thickness less than 2 mm.

### IV. Wound Closure

In one aspect, the present disclosure provides a method for closing an acute wound comprising applying a skin substitute over an acute wound, wherein the skin substitute is an organotypic human skin equivalent comprising NIKS cells. The skin substitute may be applied to the wound within hours or days of the wound's formation, within the first or second week of the wound's formation, or even later. When the total surface area of the wound is less than the manufactured size of a skin substitute, the skin substitute may be trimmed to fit the surface area and shape of the burn. When the total surface area of the wound is greater than the manufactured size of a skin substitute, multiple samples may be used to cover the wound area by abutting the multiple samples. For the avoidance of doubt, the use of multiple samples to completely cover a wound area is considered a single application. In some embodiments, only a single application of the skin substitute is needed. In other embodiments, the method comprises multiple applications of the skin substitute to the wound or a portion of the wound on different days. The timing of each subsequent application may vary. For instance a subsequent (e.g., 2^{nd}, 3^{rd}, 4^{th} etc.) application may occur days or weeks after the preceding application. In all embodiments, the application of allogenic tissue, autologous tissue or autologous cells over or under the skin substitute is not needed for wound closure. However, depending upon the total surface area of the wound, allogenic tissue, autologous tissue, autologous cells, or a different skin substitute may be used in conjunction with the skin substitute in some embodiments. For example, a wound area may be covered by abutting one or more samples of the skin substitutes and one or more autologous tissue grafts. In addition, it may be advantageous to introduce a source of autologous keratinocytes or autologous keratinocytes precursor cells to the wound area to speed-up wound healing—for example by incoporating the cells into the substitute, applying the cells over or under the skin substitutes, or using a small autologous tissue graft—when the acute wound is a full thickness wounds with a large surface area in order. Wounds are typically prepared for treatment by methods known in the art, for instance cleansing with soap and water, surgical excision (fascial excision or tangential excision of nonviable tissue) if clinically indicated and feasible, etc. The location of the wound is not limiting. For instance, a wound may be on an arm, a leg, a torso, a face, a head, a neck, a hand, a foot, a buttock, over joints, etc.

As used herein, the term “wound closure” means ≥95% re-epithelialization of the wound surface with absence of drainage. In some embodiments, wound closure may be 95%, 96%, 97%, 98%, or 99% re-epithelialization of the wound surface with absence of drainage. In another embodiment, wound closure may be 100% re-epithelialization of the wound surface with absence of drainage. The amount of time it takes to achieve wound closure is assessed from the first application of the skin substitute (i.e., day 0). In some embodiments, wound closure may occur 3 months after the first application of the skin substitute. In some embodiments, wound closure may occur within about 1 week, about 2 weeks, about 3 weeks, about 4 weeks, about 1 month, about 2 months, or about 3 months after the first application of the skin substitute.

In still further embodiments, methods of the present disclosure for closing an acute wound may result in durable wound closure. As used herein, the term “durable wound closure” means 100% re-epithelialization of the wound surface with without drainage, confirmed by a medical provider at two visits 2-20 weeks apart. In some examples, durable wound closure occurs ≤3 months after the first application of the skin substitute. In some examples, durable wound closure occurs ≤2 months after the first application of the skin substitute. In some examples, durable wound closure occurs ≤4 weeks after the first application of the skin substitute. In some examples, durable wound closure occurs about 3 weeks after the first application of the skin substitute.

Exemplary acute wounds are detailed in Section II, and incorporated into this section by reference. In some embodiments, the acute wound is a full-thickness wound, a partial-thickness wound, or a complex wound that contains intact dermal elements and the wound type is a surgical wound, a burn wound, a bite wound, a puncture wound, a laceration or a wound from a sharp cut. In some embodiments, the acute wound a partial-thickness burn wound, or a complex burn wound that contains intact dermal elements. In some embodiments, the acute wound a partial-thickness thermal burn wound, or a complex thermal burn wound that contains intact dermal elements. In some embodiments, the acute wound a partial-thickness surgical wound, or a complex surgical wound that contains intact dermal elements. Typically, in each of the above embodiments, the wound is a debrided wound where autografting is clinically indicated, and the depth of the wound is assessed at the time application.

Suitable subjects are detailed in Section III, and incorporated into this section by reference. In some embodiments, the subject is 18-60 years of age, 18-64 years of age, or 18-65 years of age. In some embodiments, the subject is less than ≥60 years of age, ≥65 years of age, or ≥70 years of age. In some embodiments, the subject is less than 18 years of age, ≤15 years of age, or ≤10 years of age. In some embodiments, the subjects is ≤5 years of age, ≤3 years of age, ≤2 years of age, or ≤1 year of age. In some embodiments, the subject has limited surface area available for donor sites. In some embodiments, harvest of donor sites is contraindicated in the subject. In some embodiments, the subject is not hemodynamically stable. In some embodiments, the subject is at risk for delayed or impaired wound healing. In some embodiments, the subject has any combination of limited surface area available for donor sites, a contraindication for harvest of donor sites, hemodynamic instability, or a risk for delayed or impaired wound healing.

Methods of the present disclosure for closing acute wounds provide improved outcomes for the subject compared to the current standard-of-care (i.e., surgical excision and autografting). In various embodiments, an improved outcome may be reduced autografting, decreased pain, decreased scarring, decreased infection rate, decreased donor site morbidity, improved quality of life, or any combination thereof, as compared to treatment with autograft(s) only, as assessed by an observer or by the subject. In certain embodiments, an improved outcome may be reduced autografting, decreased pain, decreased scarring, improved pigmentation or coloring at a graft site, or any combination thereof. In certain embodiments, an improved outcome may be reduced autografting and/or decreased pain. Further details are provided in Section IV.

In an exemplary embodiment, the present disclosure provides a method for closing an acute wound of a subject, the method comprising applying a skin substitute over the wound, wherein the skin substitute is an organotypic human skin equivalent comprising NIKS cells, and wherein autografting is clinically indicated for the wound and the wound is a partial thickness burn wound or a complex burn wound that contains intact dermal elements. In various embodiments, the subject may have limited surface area available for a donor site, be contraindicated for autografting, be hemodynamically unstable, be at risk for delayed or impaired wound healing, or any combination thereof. In still further embodiments, the burn may be a thermal burn. In still further embodiments, autografting is decreased by at least about 25%, 50%, 75%, 90%, or 95%, or more preferably, no autografting is needed. In certain exemplary embodiments of the above, the method comprises only a single application of the skin substitute.

In another exemplary embodiment, the present disclosure provides a method for closing an acute wound of a subject, the method comprising applying a skin substitute over the wound, wherein the skin substitute is an organotypic human skin equivalent comprising NIKS cells, and wherein autografting is clinically indicated for the wound, the wound is a partial thickness burn wound or a complex burn wound that contains intact dermal elements, and the % TBSA is ≥15%, ≥20%, or ≥25% and/or the surface of the wound is ≥200 cm², ≥300 cm², ≥400 cm², or ≥500 cm². In various embodiments, the subject may have limited surface area available for a donor site, be contraindicated for autografting, be hemodynamically unstable, be at risk for delayed or impaired wound healing, or any combination thereof. In still further embodiments, the burn may be a thermal burn. In still further embodiments, autografting is decreased by at least about 25%, 50%, 75%, 90%, or 95%, or more preferably, no autografting is needed. In certain exemplary embodiments of the above, the method comprises only a single application of the skin substitute.

In another exemplary embodiment, the present disclosure provides a method for closing an acute wound of a subject, the method comprising applying a skin substitute over the wound, wherein the skin substitute is an organotypic human skin equivalent comprising NIKS cells, and wherein autografting is clinically indicated for the wound, the wound is a partial thickness burn wound or a complex burn wound that contains intact dermal elements, and the % TBSA is ≥25%, ≥35%, or ≥50% and/or the surface of the wound is ≥900 cm², ≥1000 cm², ≥2000 cm², ≥5000 cm², or ≥10,000 cm². In various embodiments, the subject may have limited surface area available for a donor site, be contraindicated for autografting, be hemodynamically unstable, be at risk for delayed or impaired wound healing, or any combination thereof. In still further embodiments, the burn may be a thermal burn. In still further embodiments, autografting is decreased by at least about 25%, 50%, 75%, 90%, or 95%, or more preferably, no autografting is needed. In certain exemplary embodiments of the above, the method comprises only a single application of the skin substitute.

In another exemplary embodiment, the present disclosure provides a method for closing an acute wound of a subject less than 18 years of age, the method comprising applying a skin substitute over the wound, wherein the skin substitute is an organotypic human skin equivalent comprising NIKS cells, and wherein autografting is clinically indicated for the wound and the wound is a partial thickness burn wound or a complex burn wound that contains intact dermal elements. In various embodiments, the subject may be about 15 years of age or less, about 10 years of age or less, about 5 years of age or less, or about 3 years of age or less. In still further embodiments, the burn may be a thermal burn. In still further embodiments, autografting is decreased by at least about 25%, 50%, 75%, 90%, or 95%, or more preferably, no autografting is needed. In certain exemplary embodiments of the above, the method comprises only a single application of the skin substitute.

In another exemplary embodiment, the present disclosure provides a method for closing an acute wound of a subject 65 years of age or greater, the method comprising applying a skin substitute over the wound, wherein the skin substitute is an organotypic human skin equivalent comprising NIKS cells, and wherein autografting is clinically indicated for the wound and the wound is a partial thickness burn wound or a complex burn wound that contains intact dermal elements. In various embodiments, the subject may have limited surface area available for a donor site and/or be at risk for delayed or impaired wound healing. In still further embodiments, the burn may be a thermal burn. In still further embodiments, autografting is decreased by at least about 25%, 50%, 75%, 90%, or 95%, or more preferably, no autografting is needed. In certain exemplary embodiments of the above, the method comprises only a single application of the skin substitute.

In another exemplary embodiment, the present disclosure provides a method for closing an acute wound of a subject 70 years of age or greater, the method comprising applying a skin substitute over the wound, wherein the skin substitute is an organotypic human skin equivalent comprising NIKS cells, and wherein autografting is clinically indicated for the wound and the wound is a partial thickness burn wound or a complex burn wound that contains intact dermal elements. In various embodiments, the subject may have limited surface area available for a donor site and/or be at risk for delayed or impaired wound healing. In still further embodiments, the burn may be a thermal burn. In still further embodiments, autografting is decreased by at least about 25%, 50%, 75%, 90%, or 95%, or more preferably, no autografting is needed. In certain exemplary embodiments of the above, the method comprises only a single application of the skin substitute.

In another exemplary embodiment, the present disclosure provides a method for closing an acute wound of a subject at risk of impaired or delayed wound healing, the method comprising applying a skin substitute over the wound, wherein the skin substitute is an organotypic human skin equivalent comprising NIKS cells, and wherein autografting is clinically indicated for the wound and the wound is a partial thickness burn wound or a complex burn wound that contains intact dermal elements. In various embodiments, the subject at risk for delayed or impaired wound healing may be a subject with thinning dermis, inhalation injury or other coexisting injury, 25% TBSA, diabetes, infection (typically at the wound site but may be elsewhere), obesity, medications known to impair wound healing, a smoking habit, excessive alcohol drinking, low blood pressure, vascular disease, edema, cancer, malnutrition, or any combination thereof. In further embodiments, the subject may be <18 years of age, ≤10 years of age, ≤5 years of age, ≤3 years of age, ≤2 years of age, ≤1 year of age, ≥60 years of age, ≥65 years of age, or ≥70 years of age. In still further embodiments, the burn may be a thermal burn. In still further embodiments, autografting is decreased by at least about 25%, 50%, 75%, 90%, or 95%, or more preferably, no autografting is needed. In certain exemplary embodiments of the above, the method comprises only a single application of the skin substitute.

In another exemplary embodiment, the present disclosure provides a method for closing an acute wound of a subject, the method comprising applying a skin substitute over the wound, wherein the skin substitute is an organotypic human skin equivalent comprising NIKS cells, and wherein autografting is clinically indicated for the wound and the wound is a partial thickness surgical wound or a complex surgical wound that contains intact dermal elements. In various embodiments, the subject may have may have limited surface area available for a donor site, be contraindicated for autografting, be hemodynamically unstable, be at risk for delayed or impaired wound healing, or any combination thereof. In still further embodiments, the surgical wound may be a skin cancer wound. In still further embodiments, autografting is decreased by at least about 25%, 50%, 75%, 90%, or 95%, or more preferably, no autografting is needed. In certain exemplary embodiments of the above, the method comprises only a single application of the skin substitute.

In another exemplary embodiment, the present disclosure provides a method for closing an acute wound of a subject less than 18 years of age, the method comprising applying a skin substitute over the wound, wherein the skin substitute is an organotypic human skin equivalent comprising NIKS cells, and wherein autografting is clinically indicated for the wound and the wound is a partial thickness surgical wound or a complex surgical wound that contains intact dermal elements. In various embodiments, the subject may be about 15 years of age or less, about 10 years of age or less, about 5 years of age or less, or about 3 years of age or less. In still further embodiments, the surgical wound may be a skin cancer wound. In still further embodiments, autografting is decreased by at least about 25%, 50%, 75%, 90%, or 95%, or more preferably, no autografting is needed. In certain exemplary embodiments of the above, the method comprises only a single application of the skin substitute.

In another exemplary embodiment, the present disclosure provides a method for closing an acute wound of a subject 65 years of age or greater, the method comprising applying a skin substitute over the wound, wherein the skin substitute is an organotypic human skin equivalent comprising NIKS cells, and wherein autografting is clinically indicated for the wound and the wound is a partial thickness surgical wound or a complex surgical wound that contains intact dermal elements. In various embodiments, the subject may have limited surface area available for a donor site and/or be at risk for delayed or impaired wound healing. In still further embodiments, the surgical wound may be a skin cancer wound. In still further embodiments, autografting is decreased by at least about 25%, 50%, 75%, 90%, or 95%, or more preferably, no autografting is needed. In certain exemplary embodiments of the above, the method comprises only a single application of the skin substitute.

In another exemplary embodiment, the present disclosure provides a method for closing an acute wound of a subject 70 years of age or greater, the method comprising applying a skin substitute over the wound, wherein the skin substitute is an organotypic human skin equivalent comprising NIKS cells, and wherein autografting is clinically indicated for the wound and the wound is a partial thickness surgical wound or a complex surgical wound that contains intact dermal elements. In various embodiments, the subject may have thinning dermis and/or be at risk for delayed or impaired wound healing. In still further embodiments, the surgical wound may be a skin cancer wound. In still further embodiments, autografting is decreased by at least about 25%, 50%, 75%, 90%, or 95%, or more preferably, no autografting is needed. In certain exemplary embodiments of the above, the method comprises only a single application of the skin substitute.

In another exemplary embodiment, the present disclosure provides a method for closing an acute wound of a subject at risk of impaired or delayed wound healing, the method comprising applying a skin substitute over the wound, wherein the skin substitute is an organotypic human skin equivalent comprising NIKS cells, and wherein autografting is clinically indicated for the wound and the wound is a partial thickness surgical wound or a complex surgical wound that contains intact dermal elements. In various embodiments, the subject at risk for delayed or impaired wound healing may be a subject with thinning dermis, ≥25% TBSA, diabetes, infection (typically at the wound site but may be elsewhere), obesity, medications known to impair wound healing, a smoking habit, excessive alcohol drinking, low blood pressure, vascular disease, edema, cancer, malnutrition, or any combination thereof. In further embodiments, the subject may be <18 years of age, ≤10 years of age, ≤5 years of age, ≤3 years of age, ≤2 years of age, ≤1 year of age, ≥60 years of age, ≥65 years of age, or ≥70 years of age. In still further embodiments, the surgical wound may be a skin cancer wound. In still further embodiments, autografting is decreased by at least about 25%, 50%, 75%, 90%, or 95%, or more preferably, no autografting is needed. In certain exemplary embodiments of the above, the method comprises only a single application of the skin substitute.

### V. Improved Outcomes

In another aspect, the present disclosure provides a method for improving an outcome of skin grafting in a subject, the method comprising applying a skin substitute over an acute wound, wherein the skin substitute is an organotypic human skin equivalent comprising NIKS cells. The skin substitute may be applied to the wound within hours or days of the wound's formation, within the first or second week of the wound's formation, or even later. When the total surface area of the wound is less than the manufactured size of a skin substitute, the skin substitute may be trimmed to fit the surface area and shape of the burn. When the total surface area of the wound is greater than the manufactured size of a skin substitute, multiple samples may be used to cover the wound area by abutting the multiple samples. For the avoidance of doubt, the use of multiple samples to completely cover a wound area is considered a single application. In some embodiments, only a single application of the skin substitute is needed. In other embodiments, the method comprises multiple applications of the skin substitute to the wound or a portion of the wound. In all embodiments, the application of cadaver tissue, autologous tissue or autologous cells over or under the skin substitute is not needed for wound closure. However, depending upon the total surface area of the wound, cadaver tissue, autologous tissue or autologous cells may be used in conjunction with the skin substitute in some embodiments. For example, a wound area may be covered by abutting one or more samples of the skin substitutes and one or more autologous tissue grafts. Wounds are typically prepared for treatment by methods known in the art, for instance cleansing with soap and water, surgical excision (fascial excision or tangential excision) if clinically indicated and feasible, etc. The location of the wound is not limiting. For instance, a wound may be on an arm, a leg, a torso, a face, a head, a neck, a hand, a foot, a buttock, over joints, etc.

In various embodiments, an improved outcome may be reduced autografting, decreased pain, decreased scarring at the donor site and/or graft site, decreased infection rate at the donor site and/or graft site, improved vascularity at the donor site and/or graft site, improved pigmentation at the donor site and/or graft site, increased pliability at the donor site and/or graft site, decreased stiffness of the tissue at the donor site and/or graft site, decreased itching at the donor site and/or graft site, decreased sensitivity to external stimuli (e.g., pain, touch, temperature, etc.) at the donor site and/or graft site, decreased donor-site morbidity, improved quality of life, or any combination thereof, as compared to treatment with autografting only, as assessed by an observer or by the subject. In certain embodiments, an improved outcome may be reduced autografting, decreased pain, decreased scarring, improved pigmentation or coloring at a graft site, or any combination thereof. In certain embodiments, an improved outcome may be reduced donor-site morbidity. Donor-site morbidities include but are not limited to tenderness, pain, cold-sensitivity, scarring in general or more particularly hypertrophic scarring, infection, conversion from a split-thickness wound to a complex wound or a full thickness wound, etc. In certain embodiments, an improved outcome may be reduced hypertrophic scarring at the donor site and/or at the graft site.

Decreased autografting is calculated by dividing the total wound area receiving an autograft as a primary intervention or after failure of a tissue substitute, by the total wound area, and subtracting that value from 1. For instance, if an autograft was applied to 2 cm² of a 10 cm² wound as a primary intervention and a tissue substitute was applied to the remaining part of the wound, there is an 80% decrease in autografting. In embodiments where an improved outcome includes decreased autografting, there may be a 1%, 5%, 10%, 15%, 20%, 25%, 30%, 35%, 40%, 50%, 55%, 60%, 70%, 75%, 80%, 85%, 90%, 95%, or 100% decrease in autografting. In certain embodiments, there may be about a 5% to about a 50% decrease in autografting, or about a 15% to about a 50% decrease in autografting, about a 25% to about a 50% decrease in autografting, or about a 35% to about a 50% decrease in autografting. In other embodiments, there may be about a 25% to about a 75% decrease in autografting, or about a 35% to about a 75% decrease in autografting, about a 45% to about a 75% decrease in autografting, or about a 55% to about a 75% decrease in autografting. In still other embodiments, there may be about a 50% to about a 100% decrease in autografting, about a 60% to about a 100% decrease in autografting, about a 70% to about a 100% decrease in autografting, or about a 80% to about a 100% decrease in autografting. In further embodiments, there may be about a 90% to 100% decrease in autografting, or even a 100% decrease in autografting.

Pain is a patient reported outcome, and may be evaluated by a variety of well-known, clinically tested pain assessment tools. Non-limiting examples include the visual analog scale and the Wong-Baker FACES pain rating scale. In an exemplary embodiment, pain is evaluated by the Wong-Baker FACES pain rating scale. One contributor to decreased pain associated with methods of the present invention is decreased autografting, which results in fewer donor sites. Decreased pain at the graft site may also contribute to the overall decrease in pain. In embodiments where an improved outcome includes decreased pain, there may be a 1%, 5%, 10%, 15%, 20%, 25%, 30%, 35%, 40%, 50%, 55%, 60%, 70%, 75%, 80%, 85%, 90%, 95%, or 100% decrease in pain. In certain embodiments, there may be about a 5% to about a 50% decrease in pain, or about a 15% to about a 50% decrease in pain, about a 25% to about a 50% decrease in pain, or about a 35% to about a 50% decrease in pain. In other embodiments, there may be about a 25% to about a 75% decrease in pain, or about a 35% to about a 75% decrease in pain, about a 45% to about a 75% decrease in pain, or about a 55% to about a 75% decrease in pain. In still other embodiments, there may be about a 50% to about a 100% decrease in pain, about a 60% to about a 100% decrease in pain, about a 70% to about a 100% decrease in pain, or about a 80% to about a 100% decrease in pain. In further embodiments, there may be about a 90% to 100% decrease in pain, or even a 100% decrease in pain.

In embodiments where an improved outcome includes decreased scarring, the evaluation may take into consideration scarring at all treatment sites, scarring at donor sites only, or scarring at graft sites only. To evaluate scarring, preferably one or more parameter of a scar is compared to normal skin on a comparable anatomic location. Non-limiting parameters include overall opinion, vascularity, pigmentation or color, thickness, relief, pliability, surface area, pain associated with a scar, and itchiness. In describing these parameters below, various tests are described which an assessor may use. However, when the evaluator is a patient, the evaluation may be subjective. Vascularity refers to the presence of vessels in scar tissue assessed by the amount of redness. It may be tested by the amount of blood return after blanching with a piece of Plexiglas (or an equivalent thereof). Pigmentation refers to brownish coloration of a scar by pigment (melanin). It may be tested by applying Plexiglas (or an equivalent thereof) to the skin with moderate pressure to eliminate the effect of vascularity. For patient assessments, “scar color” is typically evaluated. Thickness refers to an average distance between the subcutical-dermal border and the epidermal surface of a scar. Relief refers to the extent to which surface irregularities are present (preferably compared with adjacent normal skin). Pliability refers to suppleness of a scar tested by wrinkling the scar between the thumb and index finger. Surface area refers to surface area of a scar in relation to the original wound area. Overall opinion refers to the overall opinion of the scar, which may or may not be based the parameters listed above. For instance, an advantage of methods of the present disclosure is that the skin substitute can be applied without extensive meshing or without meshing at all. In contrast, it is routine in the art to mesh autografts in order to maximize the surface area that can be covered with a given piece of tissue, which in turn limits the number of donor sites and associated donor-site morbidities. Meshing, however, can compromise the final cosmetic appearance, and any negative impact may be reflected in an overall opinion score. In an exemplary embodiment, The Patient and Observer Scar Assessment Scale (POSAS) is used evaluate scarring. In embodiments where the improved outcome is decreased scarring, there may be a 1%, 5%, 10%, 15%, 20%, 25%, 30%, 35%, 40%, 50%, 55%, 60%, 70%, 75%, 80%, 85%, 90%, 95%, or 100% decrease in scarring. In certain embodiments, there may be about a 5% to about a 50% decrease in scarring, or about a 15% to about a 50% decrease in scarring, about a 25% to about a 50% decrease in scarring, or about a 35% to about a 50% decrease in scarring. In other embodiments, there may be about a 25% to about a 75% decrease in scarring, or about a 35% to about a 75% decrease in scarring, about a 45% to about a 75% decrease in scarring, or about a 55% to about a 75% decrease in scarring. In still other embodiments, there may be about a 50% to about a 100% decrease in scarring, about a 60% to about a 100% decrease in scarring, about a 70% to about a 100% decrease in scarring, or about a 80% to about a 100% decrease in scarring. In further embodiments, there may be about a 90% to 100% decrease in scarring, or even a 100% decrease in scarring.

In embodiments where the improved outcome is decreased infection, there may be a 1%, 5%, 10%, 15%, 20%, 25%, 30%, 35%, 40%, 50%, 55%, 60%, 70%, 75%, 80%, 85%, 90%, 95%, or 100% decrease in infection. The evaluation may take into consideration infection rate at all treatment sites, at donor sites only, or at graft sites only. In certain embodiments, there may be about a 1% to about a 25% decrease in infection, about a 1% to about a 15% decrease in infection, about a 1% to about a 10% decrease in infection, or about a 1% to about a 5% decrease in infection. In other embodiments, there may be about a 5% to about a 50% decrease in infection, or about a 15% to about a 50% decrease in infection, about a 25% to about a 50% decrease in infection, or about a 35% to about a 50% decrease in infection. In other embodiments, there may be about a 25% to about a 75% decrease in infection, or about a 35% to about a 75% decrease in infection, about a 45% to about a 75% decrease in infection, or about a 55% to about a 75% decrease in infection. In still other embodiments, there may be about a 50% to about a 100% decrease in infection, about a 60% to about a 100% decrease in infection, about a 70% to about a 100% decrease in infection, or about a 80% to about a 100% decrease in infection. In further embodiments, there may be about a 90% to 100% decrease in infection, or even a 100% decrease in infection. One contributor to decreased infection associated with methods of the present invention is decreased autografting, which results in fewer donor sites (i.e., open wounds). Decreased infection at the graft site may also contribute to the overall decrease in infection rate.

Quality of life is a patient reported outcome, and may be evaluated by a variety of well-known, clinically tested burn specific or generic assessment tools. Non-limiting examples include the Sickness Impact Profile, the Burn Specific Health Scale-Brief (BSHS-B), the Medical Outcome Study Short Form-36 items (SH-36), the EuroQol five dimensions questionnaire (EQ-5D), Burn Specific Health Scale-Abbreviated (BSH-A), Burn Specific Health Scale-Revised 15D (BSHS-R), Quality of Life Questionnaire (QLQ), and the like. In embodiments where an improved outcome includes increased quality of life (QOL), there may be a 1%, 5%, 10%, 15%, 20%, 25%, 30%, 35%, 40%, 50%, 55%, 60%, 70%, 75%, 80%, 85%, 90%, 95%, 100% or more increase in the QOL score. In certain embodiments, there may be about a 5% to about a 50% increase in QOL score, or about a 15% to about a 50% increase in QOL score, about a 25% to about a 50% increase in QOL score, or about a 35% to about a 50% increase in QOL score. In other embodiments, there may be about a 25% to about a 75% increase in QOL score, or about a 35% to about a 75% increase in QOL score, about a 45% to about a 75% increase in QOL score, or about a 55% to about a 75% increase in QOL score. In still other embodiments, there may be about a 50% to about a 100% increase in QOL score, about a 60% to about a 100% increase in QOL score, about a 70% to about a 100% increase in QOL score, or about a 80% to about a 100% increase in QOL score. In further embodiments, there may be about a 90% to 100% increase in QOL score, or even a 100% increase in QOL score.

Exemplary acute wounds are detailed in Section II, and incorporated into this section by reference. In some embodiments, the acute wound is a full-thickness wound, a partial-thickness wound, or a complex wound that contains intact dermal elements and the wound type is a surgical wound, a burn wound, a bite wound, a puncture wound, a laceration or a wound from a sharp cut. In some embodiments, the acute wound a partial-thickness burn wound, or a complex burn wound that contains intact dermal elements. In some embodiments, the acute wound a partial-thickness thermal burn wound, or a complex thermal burn wound that contains intact dermal elements. In some embodiments, the acute wound a partial-thickness surgical wound, or a complex surgical wound that contains intact dermal elements. Typically, in each of the above embodiments, the wound is a debrided wound where autografting is clinically indicated.

Suitable subjects are detailed in Section III, and incorporated into this section by reference. In some embodiments, the subject is 18-60 years of age, 18-64 years of age, or 18-65 years of age. In some embodiments, the subject is less than ≥60 years of age, or ≥65 years of age. In some embodiments, the subject is less than 18 years of age, ≤15 years of age, or ≤10 years of age. In some embodiments, the subjects is ≤5 years of age, ≤3 years of age, ≤2 years of age, or ≤1 year of age. In some embodiments, the subject has limited surface area available for donor sites (e.g., young, elderly, ≥25% TBSA, etc.). In some embodiments, harvest of donor sites is contraindicated in the subject. In some embodiments, the subject is not hemodynamically stable. In some embodiments, the subject is at risk for delayed or impaired wound healing. In some embodiments, the subject has any combination of limited surface area available for donor sites, a contraindication for harvest of donor sites, hemodynamic instability, or a risk for delayed or impaired wound healing.

So that the present invention may be more readily understood, certain terms are defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

The term “about,” as used herein, refers to variation of in the numerical quantity that can occur, for example, through typical measuring techniques and equipment, with respect to any quantifiable variable, including, but not limited to, mass, volume, time, distance, and amount. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations, which can be up to ±5%, but can also be ±4%, 3%, 2%, 1%, etc. Whether or not modified by the term “about,” the claims include equivalents to the quantities.

### EXAMPLES

The following examples illustrate various iterations of the invention.

### Example 1

To evaluate whether StrataGraft could be used to reduce the need for pain in complex thermal burn wounds that contain dermal elements (“DPT thermal burns”), the skin substitute was evaluated in a prospective, randomized, controlled, open-label study that assessed the safety, tolerability, and efficacy of increasing amounts of a single application of StrataGraft compared to autograft.

The study was conducted with 3 cohorts at 6 burn centers in the United States. Subjects were sequentially enrolled into the first 2 cohorts, stratified by maximum-allowed treatment-area size, and received StrataGraft tissue that had been stored refrigerated (2° C.-8° C.). Cohorts 1 and 2 were treated with 220 cm² and 440 cm² of StrataGraft tissue, respectively. The third cohort was treated with 440 cm² of StrataGraft tissue that was cryopreserved (stored at −70° C. to −90° C.) and thawed just prior to application. Target wound size was determined as a factor of the production size of StrataGraft tissue (44 cm²) and dose (i.e., size) escalation strategy. An intrapatient comparator accounted for subject-to-subject variability in underlying physiologies, healing trajectories, patient-specific characteristics, and scarring. Two areas of comparable DPT burn on each patient (contiguous or noncontiguous) on the arms, legs, or torso, were randomly assigned 1:1 to receive StrataGraft tissue or autograft control treatment. Randomization schemes were generated using a customized program (ResearchPoint Global, Austin, Tex.), prior to study initiation. A sealed randomization envelope was provided for use with each subject. None of the clinical site personnel were informed of treatment-site randomization assignments until wound excision and confirmation of eligibility were complete. To maximize the amount of StrataGraft tissue applied, the StrataGraft tissue treatment area in cohorts 2 and 3 could be up to twice that of the autograft control.

Study-site personnel were trained on the appropriate handling of StrataGraft tissue prior to site activation. The study surgical procedures such as tangential excision, autograft harvest, and graft placement are part of the standard of care for burn wounds. Wound-bed preparation and graft anchoring were performed similarly for both treatment sites.

Eligible subjects were aged 18-65 years with thermal burns of 3%-49% total body surface area (TBSA), including areas of DPT burn of 88-880 cm² that underwent excision and pain, and who also had sufficient healthy skin designated as donor sites for the 2 study-treatment sites. Subjects with insulin dependent diabetes mellitus prior to admission, a history of malignancy or who were receiving systemic immunosuppressive therapy, or concurrent conditions that could compromise safety were excluded from study participation. Treatment sites excluded were: FT burns; chronic wounds; sites adjacent to unexcised eschar; wounds on the face, head, neck, hands, feet, buttocks, and over joints; and wounds deemed clinically infected.

Two areas of uninjured skin were prospectively designated as donor sites to provide autograft tissue for the control site and, if needed, for the StrataGraft tissue treatment site. The donor site prospectively identified for the StrataGraft tissue treatment site was reserved in the event that salvage grafting was undertaken for the StrataGraft tissue treatment site. Both StrataGraft tissue and autograft were applied following surgical excision of nonviable tissue from the burn and establishment of hemostasis. Autograft was harvested from the identified donor site for placement on the autograft site. Both StrataGraft tissue and autograft were meshed 1:1, trimmed to fit the wound, and secured with surgical staples. Wounds outside of study treatment sites were managed according to the institutional standard of care.

Porous high-density polyethylene primary dressings were applied in the operating room and removed after 7±1 days, per clinician judgment. Secondary dressings of bismuth-impregnated petroleum gauze followed by dry gauze and an additional outer immobilizing dressing, at the discretion of investigator, were applied in the operating room and changed on Days 3, 7±1, 14±2 28±3, and then as needed. Donor-site foam dressings, dry gauze, and an additional outer immobilizing dressing (at the discretion of the investigator) were applied in the operating room and removed by Day 7, and wounds were then covered with bismuth-impregnated petroleum gauze until healed.

Coprimary end points were the percent area of the StrataGraft treatment site autografted by Day 28 and wound closure (defined as ≥95% re-epithelialization with absence of drainage) of the treatment sites at 3 months after treatment. Secondary efficacy end points included the proportion of treatment-site wounds completely closed, percent wound closure, cosmesis of treatment and donor sites, and donor-site pain. Safety end points included the monitoring of AEs, serious AEs (SAEs), incidence of infection, vital signs, and hematologic parameters. The presence of allogeneic DNA from StrataGraft was assayed using a forensic genotyping kit (Powerplex 16, Promega, Madison Wis.; University of Wisconsin Hospital and Clinics Molecular Diagnostics Laboratory, Madison, Wis.). Blood collected prior to StrataGraft treatment served as a patient-specific control for comparison with DNA isolated from a biopsy taken from the StrataGraft treatment site at 3 months. The sensitivity limit for detection of short tandem-repeat polymorphisms unique to StrataGraft was 2%.

Subjects were observed for 12 months, with study-related visits occurring at Day 0 (time of placement), Days 3, 7±1, 14±2, 28±3, and Months 3±14 days, as well as Months 6±1 and 12±1 after treatment. Clinicians assessed treatment-site appearance and determined the area of StrataGraft treatment sites autografted at Days 3, 7±1, 14±2, and 28±3. Donor site pain was assessed at Days 3, 7±1, 14±2, and 28±3 using the Wong-Baker FACES1 pain-rating scale in which 6 visual categories are converted into corresponding numerical scores for comparison: from “no pain” (score of 0) to “worst pain” (score of 5). Wound closure of treatment sites was assessed at Days 7±1 14±2, and 28±3 and at Months 3±14 days, 6±1 month, and 12±1 month. Cosmesis of treatment and donor sites was assessed by both clinical observers and subjects at Months 3±14 days, 6±1 month, and 12±1 month using the validated Patient and Observer Scar Assessment Scale (POSAS), version 2.0. Subjects and observers separately assessed 6 cosmetic categories and an overall opinion, each ranked on a scale from 1 (normal skin) to 10 (worst imaginable). Category scores were summed to create a total score from each assessor.

Observer assessments included categories of vascularity, pigmentation, thickness, relief, pliability, and surface area, as well as an overall opinion score. Total scores for observer assessments were derived from the summation of categories, each ranked on a scale of 1 (normal skin) to 10 (worst scar imaginable). Patient assessments included categories of pain, itching, color, stiffness, thickness, and irregularity, as well as an overall opinion score. Total scores for patient assessments were derived from the summation of categories, ranked for pain and itching categories on a scale of 1 (no, not at all) to 10 (yes, very much), and on a scale of 1 (normal skin) to 10 (very different) for the remaining categories. The overall opinion scores were separately ranked on a scale of 1 (normal skin) to 10 (“worst scar imaginable” for observer assessment and “very different” for patient assessment

Sample size was limited due to the rarity of the condition and included consideration of the precision (the width of a 2-sided approximate 95% confidence interval [CI]) for estimating the percentage of subjects spared autograft harvest. At P=0.5, the 95% CI for a population of 30 would be 50%±16.8%. This study was not prospectively powered to detect statistically significant differences between treatment groups, therefore descriptive statistics were used. Fisher's Exact Test was used to compare between treatment groups the proportion of wounds that achieved wound closure at 3 months. Differences in wound-closure rates between treatment groups across cohorts were determined using the Cochran-Mantel-Haenszel Test. Differences in re-epithelialization within each subject between treatment groups by cohort were determined using the Wilcoxon Signed-Rank Test. Post hoc Wilcoxon Signed-Rank analysis determined differences in donor site pain between treatment groups. Total POSAS score and overall opinion were compared between treatment groups using the Wilcoxon Signed-Rank test. Statistical significance for all tests was declared at P<0.05. Analyses were performed in SAS, version 9.4 (SAS Institute, Inc., Cary, N.C.).

The intent-to-treat (ITT) population included all subjects who received StrataGraft tissue, regardless of amount or follow-up status. The per-protocol population included all subjects who received any amount of StrataGraft tissue and did not have a major protocol violation.

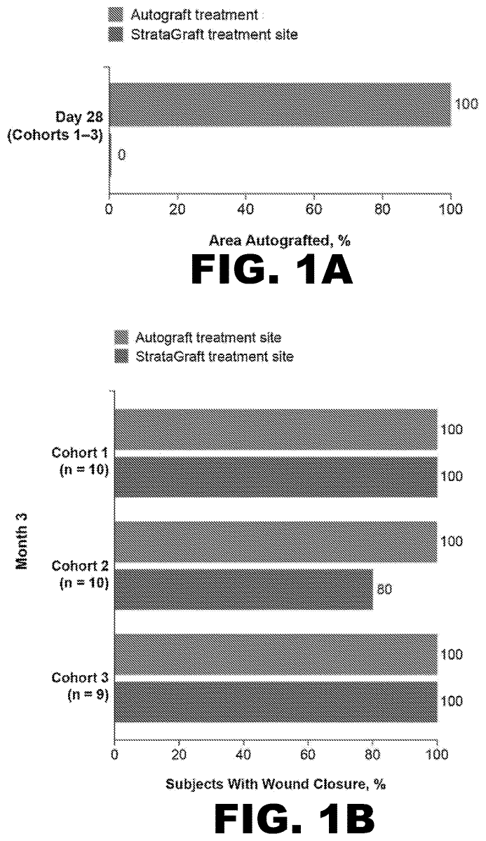

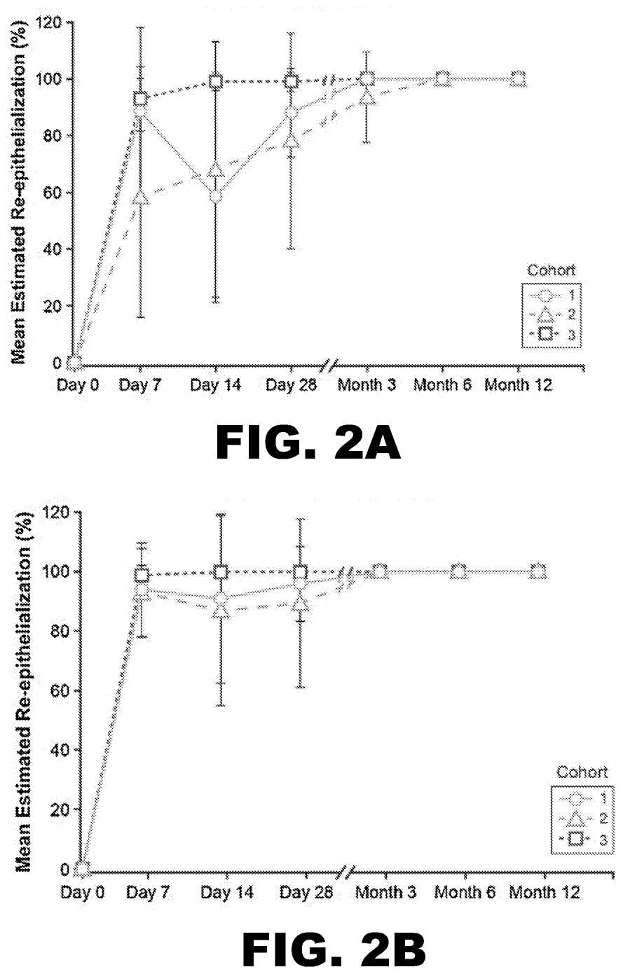

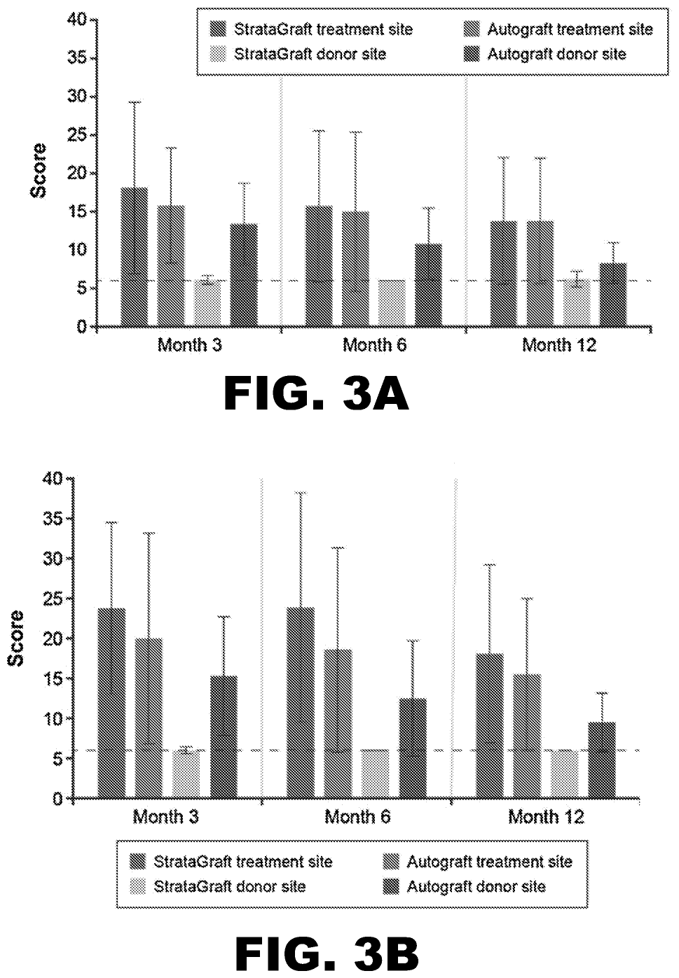

Subject demographics and baseline characteristics are provided in Table 1. Results are provided in Table 2 and FIG. 1-3, and summarized briefly below. See also, Holmes et al Burns 2019, the disclosures of which are incorporated herein by reference.

This study evaluated StrataGraft compared to autograft for the treatment of DPT thermal burns. No subjects underwent subsequent pain at the StrataGraft treatment site by Day 28. In a recent survey (data not shown), the majority of burn physicians stated that a 50% reduction in the area of DPT burns that needed pain would be regarded as a clinically significant advance in the treatment of thermal burns. The results of this study indicate that a single application of StrataGraft promoted wound closure at a frequency comparable to pain, with closure of the StrataGraft treatment sites achieved by Month 3 for 93% of subjects. Importantly, the significant reduction in donor sites by treatment with StrataGraft reduced pain and other donor site morbidities.

Mean re-epithelialization of StrataGraft treatment sites at 2 weeks lagged behind those of autograft treatment sites; however, by Day 28, no statistically significant differences were observed. Different mechanisms of healing likely contributed to the observed differences. Autografts represent autologous tissue that is transplanted and intended to engraft. After several days, autologous blood vessels in an autograft undergo anastomosis with those of the underlying tissue, promoting engraftment. The interstices of expanded meshed autograft typically generate scar tissue and produce a meshed pattern that may chronically persist. In contrast to autograft, StrataGraft likely stimulates autologous cells to regenerate a stratified squamous epithelium, a process that requires recruitment, proliferation, and organization of epidermal cells. Allogeneic DNA from StrataGraft was not detectable 3 months after treatment, consistent with the healing of StrataGraft treatment sites by the subjects' own cells and displacement of StrataGraft during wound healing. Further, it supports the proposed mechanism of action of autologous tissue regeneration in StrataGraft-treated DPT burns. In addition, because StrataGraft does not heal by engraftment, no mesh pattern was observed in the healed StrataGraft treatment sites in this study.

The StrataGraft safety profile was comparable to that of autograft. Pruritus was the most frequently reported TEAE (5 subjects), with 2 of these listed as possibly or probably related to StrataGraft treatment, and all other TEAEs were reported by 3 (10%) or fewer subjects. All treatment-related TEAEs were mild or moderate. SAEs occurred in 6 (20%) subjects, all of which resolved. Only 1 SAE was considered related to StrataGraft treatment and was associated with treatment of a wound that failed to meet study-inclusion criteria.

Given that visible scarring, deformities, contractures, and other changes in skin following burn injury can have a long-lasting negative impact on quality of life and self-esteem, cosmesis is an important consideration. In this study, cosmesis of StrataGraft treatment sites was similar to that of autograft treatment sites.

Evaluation of scarring using POSAS scores demonstrated no significant differences between outcomes for StrataGraft and autograft treatment sites at any assessed timepoint (observers) or at 12 months after treatment (subjects). Unsurprisingly, cosmesis for the prospectively identified, unharvested, StrataGraft donor sites (reserved in the event that salvage grafting of StrataGraft treatment sites was undertaken) was generally rated more favorably than surgically harvested autograft donor sites. Subjects also reported lower mean pain scores at the prospectively identified StrataGraft donor sites compared with the harvested autograft donor sites from Day 3 through Day 28, including significantly lower pain scores on Day 7. As no subjects received an autograft at the StrataGraft treatment site by Day 28, StrataGraft treatment eliminated the harvest of donor skin and associated acute pain in all subjects at that timepoint. Wound closure was achieved with both refrigerated and cryopreserved StrataGraft. Cryopreservation extends the shelf-life of StrataGraft approximately 50-fold, from 8 days to 12 months (data not shown).

TABLE 1
Subject demographics and baseline characteristics

| | Cohort 1 ^{b} | | Cohort 2 ^{c} | | Cohort 3 ^{d} | | Overall | |
|---|---|---|---|---|---|---|---|---|
| Parameter | (n = 10) | | (n = 10) | | (n = 10) | | (n = 30) | |
| Age, years | 39.7 | (11.5) | 42.1 | (13.1) | 41.3 | (12.9) | 41.0 | (12.1) |
| Sex, n (%) | | | | | | | | |
| Male | 7 | (70.0) | 5 | (0.0) | 9 | (90.0) | 21 | (70.0) |
| Female | 3 | (30.0) | 5 | (50.0) | 1 | (10.0) | 9 | (30.0) |
| Race, n (%) | | | | | | | | |
| White | 9 | (90.0) | 9 | (90.0) | 10 | (100.0) | 28 | (99.3) |
| Black or African American | 1 | (10.0) | 1 | (10.0) | 0 | (0.0) | 2 | (6.7) |
| Time from burn to graft placement | 7.9 | (3.4) | 6.9 | (3.0) | 6.8 | (2.9) | 7.3 | (3.0) |
| (days) | | | | | | | | |
| Total body surface area (%) | 9.2 | (5.9) | 16.0 | (9.4) | 16.5 | (12.6) | 13.9 | (10.0) |
| burned ^{a} | | | | | | | | |
| SG treatment area (cm²) | 112 | (46.8) | 295 | (104.6) | 262 | (145.0) | 223 | (131.1) |
| (minimum, maximum) | (52, 216) | | (150, 440) | | (78, 440) | | (52, 440) | |
| AG treatment area (cm²) | 110 | (42.9) | 183 | (67.5) | 190 | (137.0) | 161 | (95.8) |

| | | | | |
|---|---|---|---|---|
| (minimum, maximum) | (65, 204) | (74, 300) | (42, 440) | (42, 440) |

Data are from the intent-to-treat population and are mean [standard deviation], unless otherwise specified.
SG = StrataGraft,
AG = Autograft
^{a} Represents the sum of second- and third-degree burns.
^{b} Eligible to receive up to 220 cm² of refrigerated SG.
^{c} Eligible to receive up to 440 cm² of refrigerated SG.
^{d} Eligible to receive up to 440 cm² of cryopreserved SG.

TABLE 2
Summary of percent re-epithelialization: ITT population

| | | | Mean (SD) % Re-epithelialization | | | |
|---|---|---|---|---|---|---|
| Timepoint | Cohort | Patients (n) | SG Site | | AG Site | |
| Day 7 | 1 | 10 | 88.5 | (29.6) | 94.0 | (15.8) |
| | 2 | 10 | 58.0 | (42.1) | 93.0 | (14.9) |
| | 3 | 10 | 93.0 | (11.4) | 99.0 | (3.2) |
| Day 14 | 1 | 10 | 58.5 | (37.4) | 91.0 | (28.5) |
| | 2 | 10 | 68.0 | (45.2) | 87.0 | (32.0) |
| | 3 | 10 | 99.0 | (3.2) | 100.0 | (0) |
| Day 28 | 1 | 10 | 88.0 | (15.7) | 96.0 | (12.7) |
| | 2 | 10 | 78.0 | (38.0) | 89.5 | (28.3) |
| | 3 | 9 | 98.9 | (3.3) | 100.0 | (0) |
| Month 3 | 1 | 10 | 100.0 | (0) | 100.0 | (0) |
| | 2 | 10 | 93.5 | (16.0) | 100.0 | (0) |
| | 3 | 9 | 100.0 | (0) | 100.0 | (0) |
| Month 6 | 1 | 9 | 100.0 | (0) | 100.0 | (0) |
| | 2 | 10 | 100.0 | (0) | 100.0 | (0) |
| | 3 | 7 | 100.0 | (0) | 100.0 | (0) |

SG = StrataGraft,
AG = Autograft,
SD = standard deviation

### Example 2

This example reports the findings of a post hoc analysis of the trial described in Example 1, reporting patient outcomes according to the size of the StrataGraft treatment area (<200 cm² vs. ≥200 cm²).

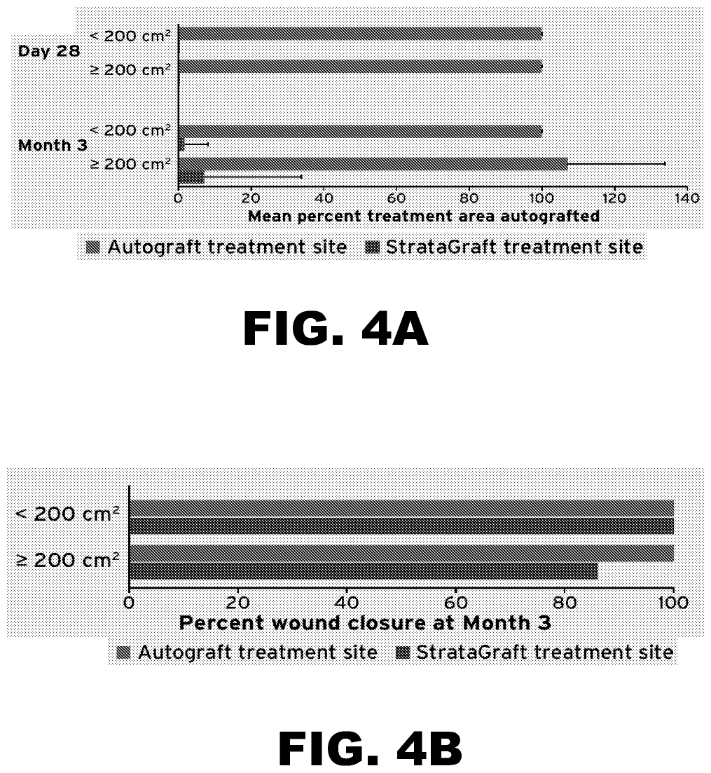

By Day 28, no subject in either treatment-sized group received autograft at the StrataGraft treatment site. By Month 3, the mean percent area of the StrataGraft treatment site that had received autograft was 1.7±6.5% and 7.1±26.7% in the <200 cm2 and ≥200 cm2 groups, respectively (FIG. 4A). In both treatment-size groups, wound closure at StrataGraft and autograft treatment sites were comparable (Table 4, FIG. 4B). The mean percent re-epithelialization of the StrataGraft treatment site did not differ significantly from that of the autograft site at Day 28 in the <200 cm² group (92.0±13.9% vs. 97.3±10.3%; P=0.31) or in the ≥200 cm² group (83.6.0±33.0% vs. 92.5±24.1%; P=0.25). Donor-site pain at prospective unharvested StrataGraft treatment sites was significantly lower than at autograft donor sites through Day 14 for subjects in both treatment-size groups (data now shown). Finally, the mean POSAS total scores (determined independently by clinical observer and subject) for StrataGraft and autograft treatment sites at Month 12 are summarized Table 5.

TABLE 3
Subject demographics and baseline characteristics
by size of treatment area: ITT population

| | Size of SG treatment area | | |
|---|---|---|---|
| | <200 cm² | ≥200 cm² | Overall |
| Parameter | (n = 15) | (n = 15) | (n = 30) |
| Age, years, | 42.0 ± 12.2 | 40.1 ± 12.4 | 41.0 ± 12.1 |
| mean ± SD (range) | (22-60) | (21-63) | (21-63) |
| Sex, n | | | |
| Male | 60 | 80 | 70 |
| Female | 40 | 20 | 30 |
| Race, n (%) | | | |
| White | 13 | 15 | 28 |
| Black or African American | 2 | 0 | 2 |
| Total body surface area (%) burned, | 10.4 ± 6.4 | 17.4 ± 11.7 | 13.9 ± 1.0 |
| mean ± SD (range) | (3-24) | (5-43) | (3-43) |
| Days from burn to tissue placement, | 7.7 ± 3.4 | 6.7 ± 2.6 | 7.2 ± 3.0 |
| mean ± SD (range) | (3-13) | (3-12) | (3-13) |
| SG treatment area (cm²), | 113.5 ± 37.1 | 332.7 ± 92.1 | 223.1 ± 131.1 |
| mean ± SD (range) | (52-176) | (204-440) | (52-440) |

ITT population
SG = StrataGraft,
SD = standard deviation

TABLE 4
Summary of wound closure: ITT population

| | | | | | | Rank- | Kruskal- |
|---|---|---|---|---|---|---|---|
| | Size of | Closed wounds, % (n) | | McNemar | Mean ± SD | sum P | Wallis P |
| Timepoint | treated area | SG site | AG site | P value | (90% CI) | value | value |
| Day 7 | <200 cm² | 60.0 (9) | 86.7 (13) | 0.10 | 0.27 ± 0.59 | 0.22 | |
| | (n = 15) | | | | (0, 0.54) | | |
| | ≥200 cm² | 53.3 (8) | 80.0 (12) | 0.05 | 0.27 ± 0.46 | 0.13 | 0.92 |
| | (n = 15) | | | | (0.06, 0.47) | | |
| Day 14 | <200 cm² | 53.3(8) | 93.3 (14) | 0.01* | 0.40 ± 0.51 | 0.03* | |
| | (n = 15) | | | | (0.17, 0.63) | | |
| | ≥200 cm² | 60.0 (9) | 86.7 (13) | 0.05 | 0.27 ± 0.46 | 0.13 | 0.45 |
| | (n = 15) | | | | (0.06, 0.47) | | |
| Day 28 | <200 cm² | 66.7 (10) | 93.3 (14) | 0.05 | 0.27 ± 0.46 | 0.13 | |
| | (n = 14)* | | | | (0.06, 0.47) | | |
| | ≥200 cm² | 71.4 (10) | 85.7 (12) | 0.16 | 0.14 ± 0.36 | 0.50 | 0.42 |
| | (n = 15) | | | | (−0.03, 0.31) | | |
| Month 3 | <200 cm² | 100 (15) | 100 (15) | — | 0 | — | |
| | (n = 15) | | | | (0) | | |
| | ≥200 cm² | 85.7 (14)^{a} | 100 (14) | — | 0.14 ± 0.36 | 0.50 | 0.14 |
| | (n = 15) | | | | (−0.03, 0.31) | | |

Significant differences (P < 0.05) are indicated by asterisks.
1 One subject missed the Day 28 study session, and was lost to follow-up by Month 3.
AG = autograft, CI = confidence interval; ITT = intent to treat, SG = StrataGraft

TABLE 5
Comesis assessment at Month 12: ITT population

| | | | | POSAS total score ^{a} | | |
|---|---|---|---|---|---|---|
| | | | | | | Kruskal- |
| | | Size of | Treatment | Value ^{a} | Rank-sum | Wallis P |
| Study site | Assessor | treated area | site | (mean ± SD) | P value | value |
| Donor | Trained | <200 cm² | SG | 6.4 ± 1.4 | <0.01* | |
| | clinical | | AG | 7.8 ± 2.4 | | 0.30 |
| | observer | ≥200 cm² | SG | 6.0 ± 0.0 | <0.01* | |
| | | | AG | 8.8 ± 2.9 | | |
| | Subject | <200 cm² | SG | 6.0 ± 0.0 | <0.01* | |
| | | | AG | 9.5 ± 3.9 | | 0.89 |
| | | ≥200 cm² | SG | 6.0 ± 0.0 | <0.01* | |
| | | | AG | 9.5 ± 3.6 | | |
| Treatment | Trained | <200 cm² | SG | 14.5 ± 8.9 | 0.68 | |
| | clinical | | AG | 13.3 ± 8.0 | | 0.92 |
| | observer | ≥200 cm² | SG | 13.0 ± 7.8 | 0.22 | |
| | | | AG | 14.2 ± 8.7 | | |
| | Subject | <200 cm² | SG | 17.5 ± 11.1 | 0.02* | |
| | | | AG | 14.5 ± 10.6 | | 0.35 |
| | | ≥200 cm² | SG | 18.7 ± 11.7 | 0.56 | |
| | | | AG | 16.5 ± 8.6 | | |

Significant differences (P < 0.05) are indicated by asterisks.
^{a} POSAS total score is derived from summation of 6 individual categories, each scored from 1 (best) to 10 (worst), yielding total scores that range from 6 (best) to 60 (worst).
AG = autograft, ITT = intent to treat, POSAS = Patient and Observer Assessment Scale, SD = Standard deviation, SG = StrataGraft

### Example 3

A phase 3 open-label randomized controlled study (NCT03005106) was performed to evaluate whether treatment with StrataGraft skin tissue can promote the healing of complex skin defects due to thermal burns that contain intact dermal elements and for which surgical excision and autografts are indicated.

Study design: The study was conducted at 12 burn centers in the United States. Randomization schemes were generated via a customized program (WuXi Clinical, Austin, Tex.) prior to the study, and randomization assignments for the treatment sites of each study patient were provided in sealed envelopes. The treated wounds were classified as deep partial thickness (DPT) thermal burns that contained intact dermal elements and were clinically indicated for excision and grafting. Following surgical excision of nonviable tissue, two comparable areas of equivalent depth were identified by the surgeon, labeled “A” and “B,” and randomly assigned to receive StrataGraft or autograft (intrapatient control). Patient eligibility was confirmed by the surgeon in the operating room after tangential excision prior to providing study-site personnel with the randomized treatment assignment. This study included an intrapatient comparator site rather than a between-patient matched control design to avoid potential variability associated with immunologic, physiologic, and healing differences that may occur in patients. Each treatment site represented a single, contiguous area.

Prior to site activation, study-site personnel received training in the correct handling of StrataGraft and were monitored by the study sponsor to ensure proficiency in StrataGraft preparation and handling. The study was conducted in accordance with Good Clinical Practice, Title 21 of the Code of Federal Regulations, the Declaration of Helsinki, and the Health Insurance Portability and Accountability Act (HIPAA). The study protocol was approved by the Institutional Review Board at each study site. Each patient or his or her legally authorized representative provided informed written consent prior to the conduct of any study-related procedures.

Eligible patients were aged 18 years who presented with thermal burns on the torso or upper or lower extremities that comprised 3-49% total body surface area (TBSA). Patients were excluded if they were undergoing current treatment with systemic immunosuppressive therapy or had a known history of malignancy, insulin-dependent diabetes, or other concurrent conditions that could compromise their safety. Full thickness (FT) burns (i.e., having no residual dermal elements after excision) or chronic wounds were excluded. Previous excision of designated treatment or control sites was an exclusion criterion. Treatment sites on the face, head, neck, hands, feet, buttocks, or areas over joints or adjacent to unexcised eschar were excluded. Infected sites were also excluded from the study.

Patients received up to 1,000 cm² of StrataGraft or autograft. The StrataGraft treatment site could be the same size or up to twice the size of the control autograft area. Identical wound-bed preparation and graft anchoring were performed for each treatment site. Following tangential excision of nonviable tissue, both StrataGraft and autograft were applied to their respective randomly assigned treatment sites.

Autograft was placed on the assigned site after meshing up to 4:1, per standard of care, and secured in place using staples, sutures, or adhesive, based on the investigator's discretion. StrataGraft was meshed 1:1, trimmed to fit the wound, and secured in place using staples, sutures, or adhesive, based on the investigator's discretion. On each patient, two areas of healthy skin were prospectively identified as donor sites to serve as sources of healthy skin for the control (autograft) treatment site, and, if required, for the StrataGraft treatment site. Wounds external to the identified study perimeter were treated according to the given institution's standard of care.

A nonadherent, porous, high-density polyethylene contact layer dressing was placed over the StrataGraft and autograft treatment sites and secured in place per the investigator's discretion. Secondary dressings consisted of a bismuth-impregnated petroleum gauze dressing, followed by a layer of dry gauze and an outer immobilizing dressing, per the investigator's discretion. Donor sites were covered with an antimicrobial foam dressing, followed by a layer of dry gauze and an outer immobilization dressing, per the investigator's discretion. Dressings were taken down to the contact layer on postoperative Day 3 and outer dressings replaced. From Day 6 and afterward, dressing application and changes were at the investigator's discretion. Silver-containing dressings and sulfamylon were not permitted to be placed on the treatment sites.

StrataGraft was manufactured by Stratatech (Madison, Wis.) in compliance with Good Manufacturing Practice (GMP) via proprietary processes. Each lot underwent proprietary testing prior to clinical use. StrataGraft was cryopreserved, shipped deep frozen on dry ice to each study site, and stored at −70° to −90° C. prior to use.

Coprimary efficacy endpoints were: 1) the difference in the percentage area of the StrataGraft-treated and autograft-treated sites that required autograft by Month 3; and 2) the proportion of patients who achieved durable wound closure of the StrataGraft treatment site by Month 3 without autograft. Durable wound closure at Month 3 was defined as complete skin re-epithelialization without drainage or dressing requirements at 2 consecutive evaluations, at least 2 weeks but no greater than 5 months apart, including or encompassing the Month 3 timepoint. Secondary endpoints included: 1) evaluation of donor-site pain through Day 14 using the Wong-Baker FACES pain rating scale¹⁷; and 2) donor-site cosmesis, as measured by the observer total Patient and Observer Scar Assessment Scale (POSAS) score at Month 3.18 Donor-site pain (grafting/regrafting) measurements were captured up to 2 weeks after initial grafting. Data collection for treatment-site cosmesis at Month 12, as measured by observer total POSAS score, is ongoing.

Safety assessments included monitoring of treatment-emergent adverse events (TEAEs), vital signs, laboratory parameters, incidence of wound infection, donor-site complications, and immunologic responses to StrataGraft. Each patient was assessed for an immune response to StrataGraft via panel reactive antigen (PRA) at baseline (prior to StrataGraft treatment), Day 28, and Month 3. PRA evaluation was performed using solid-phase Luminex assays for CTHLA class I antibody and CL1 RFX HLA class II antibody. Positive class I antibody screens were reflexed using HLA class I C1q. For each patient, samples from all three time points were tested within the same batch. Patients with any PRA value >0% were considered to have a positive PRA response. Changes in immune status were monitored by comparing them with baseline values for each patient. At Month 3, patients had a 2 mm biopsy punch taken from the central region of the StrataGraft treatment site. Tissue samples were frozen, tested for the presence of allogeneic DNA by PCR of short tandem repeats, and compared to results from blood reference samples acquired at baseline. As StrataGraft is manufactured with media-containing bovine serum albumin (BSA), blood samples were evaluated at baseline and Month 3 for antibodies against BSA.

The percentage area of treatment sites that were autografted was compared between the StrataGraft-treated and autograft-treated sites using the Wilcoxon signed-rank test. Statistical significance was defined as one sided with 0.025 type I error. A 95% confidence interval (CI) was calculated for the mean percentage area autografted, based on the t-distribution for each treatment site and for the difference between treatment sites. The proportion of patients with StrataGraft-treated burns that achieved durable wound closure by Month 3 without autograft were calculated, with significance confirmed if the lower limit of the 95% CI was ≥50%. The difference between the mean pain scores for the designated StrataGraft and autograft donor sites through Day 14 was analyzed using a paired Student's t test. The difference between the total observer POSAS scores for StrataGraft donor site and autograft donor site was analyzed using a Student's t test.

The intention-to-treat (ITT) population consisted of all patients who were randomly assigned. All efficacy analyses were performed on a modified ITT (mITT) population, which included all treatment areas on an “as treated” basis, rather than an “as randomized” basis, in the event that randomization was not followed or the donor site was changed at the time of harvest from that which was preidentified. The safety population consisted of all patients who received any StrataGraft skin tissue, irrespective of follow-up status (i.e., all study patients whose wounds were randomized to receive StrataGraft). The per-protocol population included all patients in the mITT population without a major protocol violation.

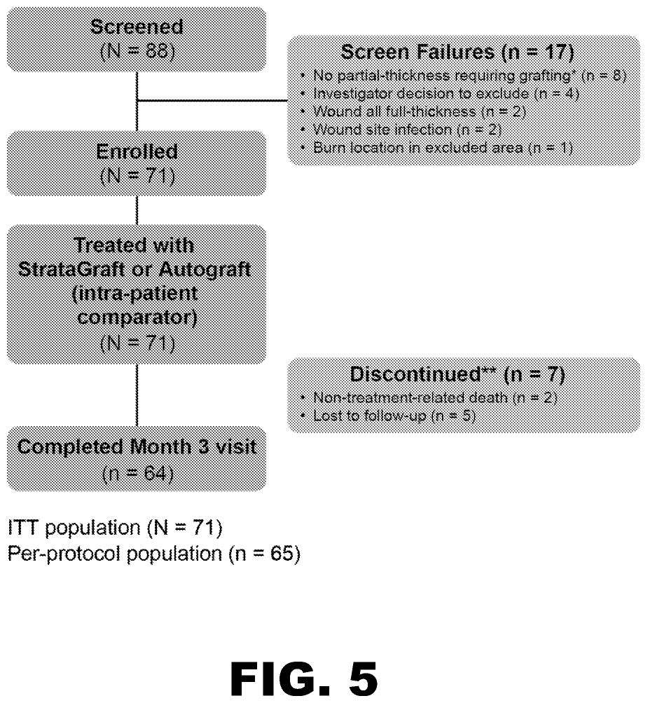

Study Results: 71 patients were enrolled (median age, 45 years [range, 19-79]; mean % TBSA, 12.0 [SD, 8.38] (FIG. 5, Table 6). Seventy-eight percent (55/71) of patients were Caucasian and 20% (14/71) were African American. As of Sep. 16, 2019, 7 patients were discontinued from the study prior to database lock for the coprimary endpoints (lost to follow-up, n=5; non-treatment-related death, n=2). The mean (SD) treatment-site area was 239.8 cm² (202.2) for StrataGraft and 219.8 cm² (233.2) for autograft. The anatomic treatment sites for StrataGraft included upper extremities (n=32), lower extremities (n=24), and torso (n=15).

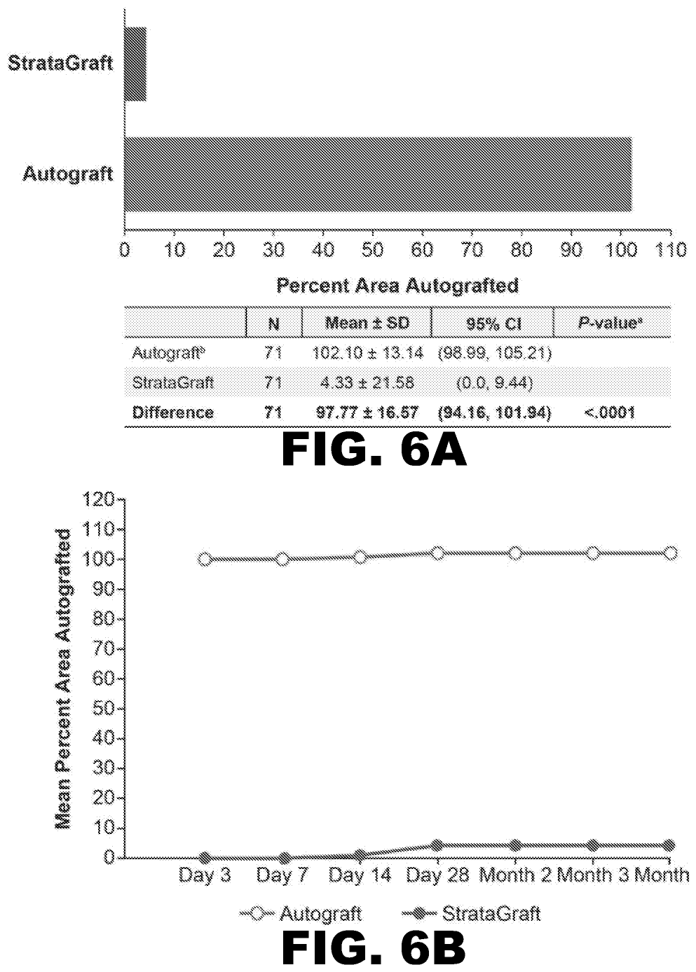

There was a significant difference in the mean percentage area of StrataGraft treatment sites that required autograft by Month 3, compared with control autograft treatment sites (4.3% vs. 102.1%, respectively; P<0.0001) (FIG. 6A). In total, 3 patients subsequently required autograft at their StrataGraft treatment site, 2 of whom also required additional autograft (regrafting) at the autograft treatment site. The mean percentage area of treatment sites that required autograft throughout the study is illustrated in FIG. 6B.

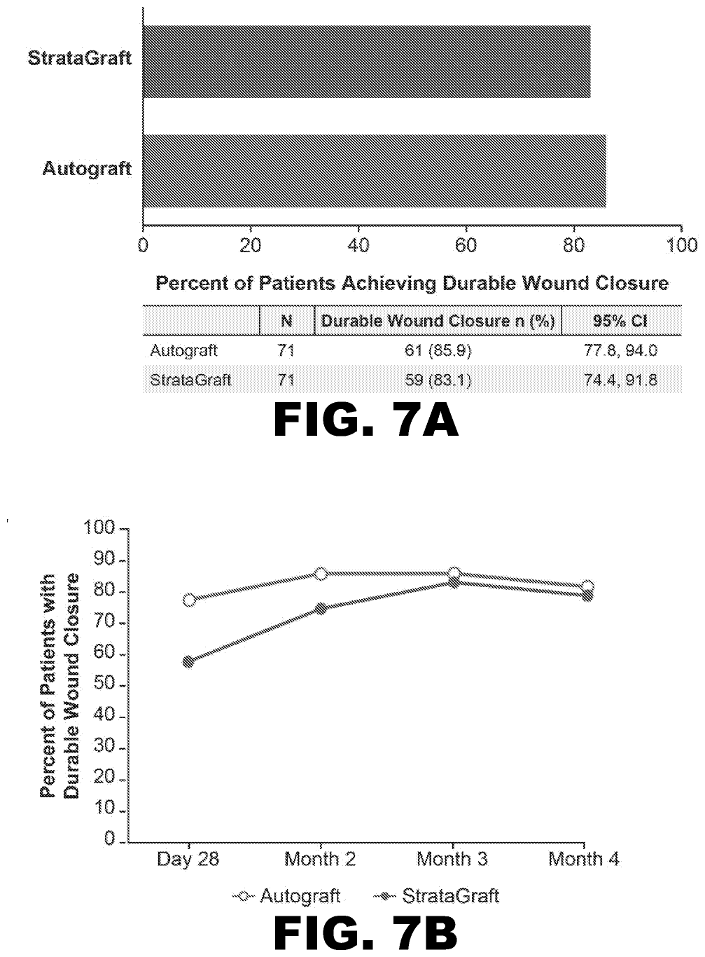

By Month 3, 83.1% (CI: 74.4, 91.8) of patients achieved durable wound closure following StrataGraft treatment (significance was predefined by the lower boundary of the 95% CI being 50%) (FIG. 7A). Following treatment with autograft, 85.9% (CI: 77.8, 94.0) of patients achieved durable wound closure (FIG. 7A). At Month 6, all StrataGraft and autograft treatment sites that met the criteria for closure at Month 3 remained closed. The percentage of patients who achieved durable wound closure without the need for autograft (at the StrataGraft treatment site) or regrafting (at the autograft treatment site) over the course of the study is summarized in FIG. 7B.

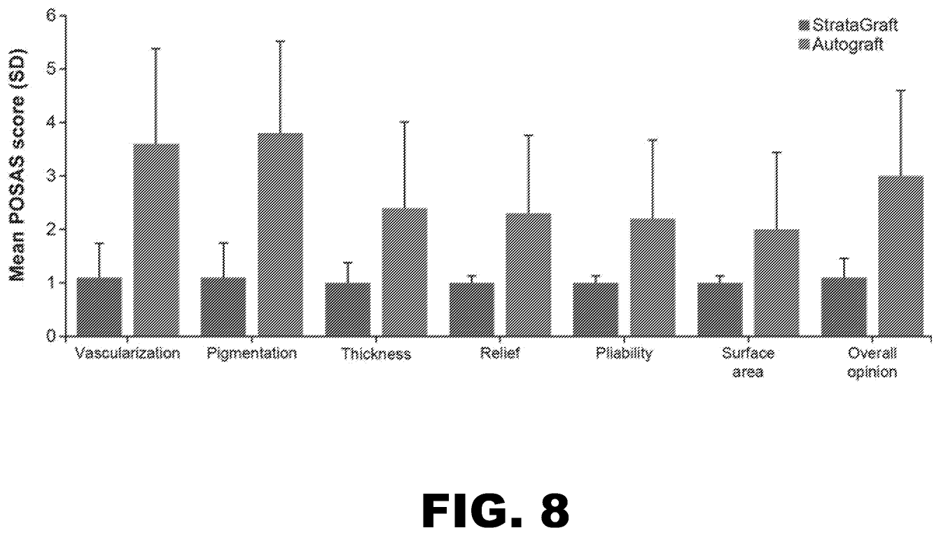

StrataGraft donor sites were prespecified and in the majority of patients, not harvested. For the 3 patients who had their StrataGraft treatment site autografted, donor-site pain was measured up to 2 weeks after initial grafting. A significant difference was observed in mean donor-site pain intensity through Day 14 between StrataGraft and autograft (0.15±0.54 vs. 2.55±1.30 respectively; P<0.0001), where a lower score is indicative of less pain. Cosmesis of donor sites at Month 3 was assessed by a clinical observer using the POSAS scale, wherein a lower score is indicative of a more favorable outcome. A score of 6 indicates healthy skin without the presence of scarring. By Month 3, mean donor-site POSAS total score was significantly lower for StrataGraft sites compared with autograft sites (6.3±1.71 vs. 16.3±7.71; P<0.0001; Table 7). Mean donor-site POSAS scores for individual scales of vascularization, pigmentation, thickness, relief, pliability, and surface area are summarized in FIG. 8. Scores for every POSAS category were lower for StrataGraft donor sites when compared to autograft donor sites.

Overall, donor-site morbidity (pain, scarring, itching, impairment of skin function) at autograft donor sites occurred in 52.1% of patients, compared with only 4.2% of patients at StrataGraft donor sites. Cosmesis at treatment sites at Month 12 is being evaluated by clinical observers using the POSAS scale.

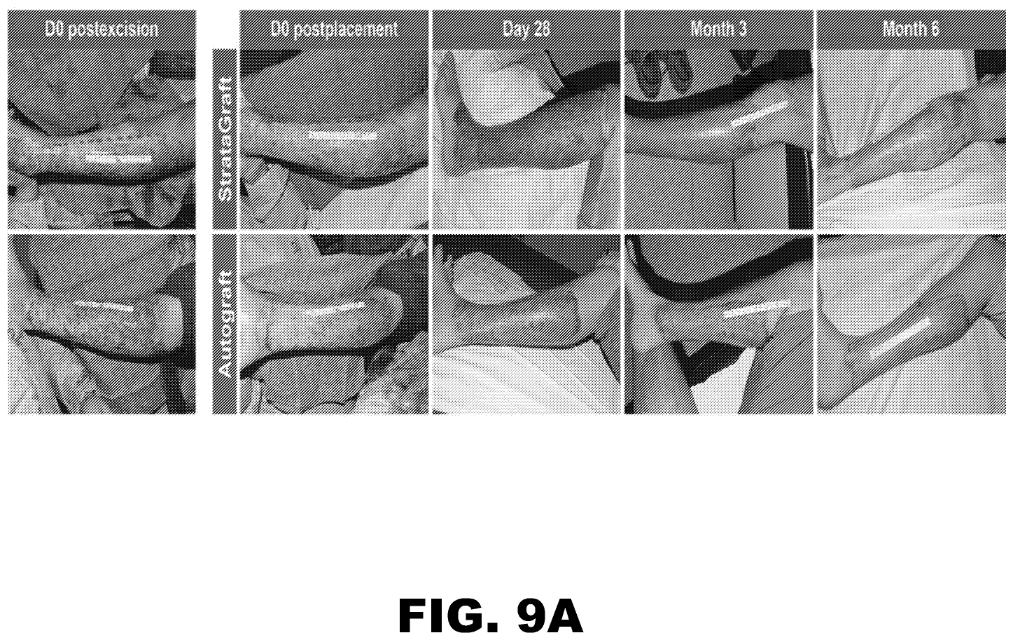

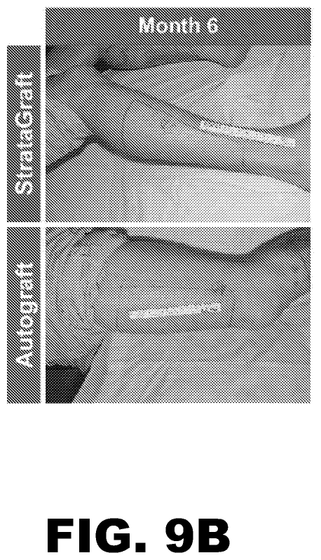

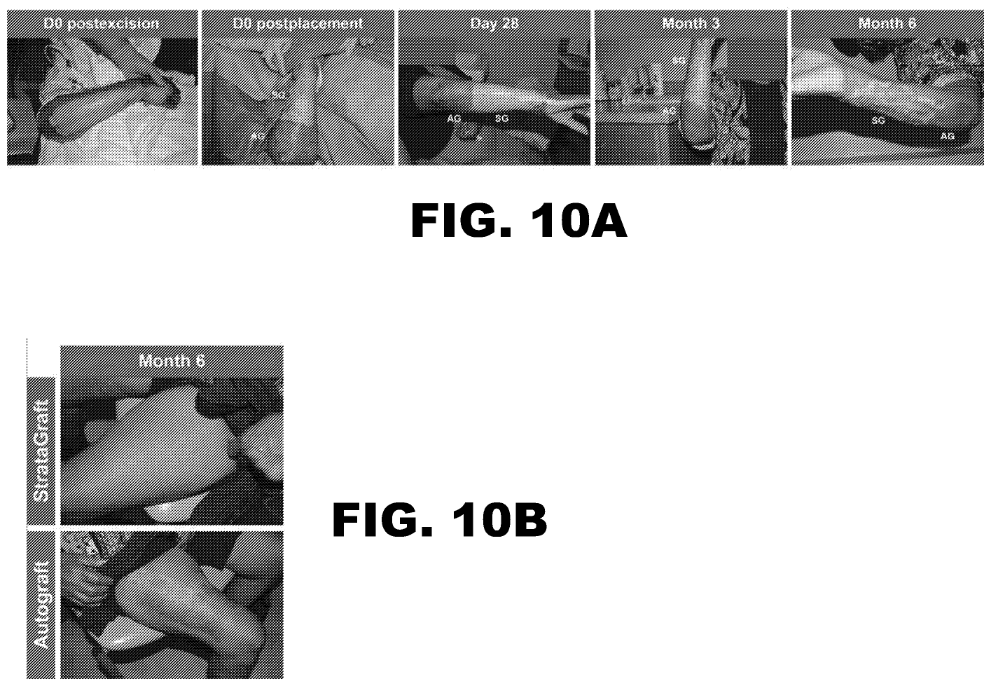

Representative photographs illustrating the wound beds for StrataGraft and autograft treatment sites and donor sites from two patients are provided (FIG. 9 and FIG. 10).

A total of 80.3% (57/71) of patients experienced at least 1 TEAE. TEAEs occurring in ≥5% of patients are summarized in Table 8. The most common TEAE was pruritus at the treatment sites, which occurred in 35.2% (25/71) of patients. Of these 25 patients, pruritus was related to StrataGraft treatment in 15.5% (11/71) of patients and not related in 19.7% (14/71) of patients, based on investigator assessment. All TEAEs related to StrataGraft treatment were mild to moderate in severity. A total of 3 (4.2%) patients had hypertrophic scar formation related to StrataGraft treatment, whereas 8 (11.3%) patients had hypertrophic scar formation that was not related to StrataGraft treatment, based on investigator assessment. Ten patients experienced TEAEs that were considered possibly related to StrataGraft treatment (hypertrophic scar, n=3; blister, n=4; excessive granulation tissue, n=2; and erythema, n=1). A total of 10 (14.1%) patients experienced at least 1 SAE-none were related to StrataGraft treatment. SAEs are summarized in Table 9 and included general disorders and administration-site conditions (n=3), cardiac disorders (n=2), infections and infestations (n=2; 1 of whom had an infection at the StrataGraft treatment site), and vascular disorders (n=2). In total, 9.9% (7/71) of patients discontinued treatment; 2 patients died (myocardial infarction, n=1; not otherwise specified, n=1, both considered unrelated to StrataGraft treatment). No patients withdrew from the study due to a TEAE.

At baseline, 4.3% (3/70) of patients had reactivity to alleles found in StrataGraft, 95.7% (67/70) of patients had reactivity to alleles not found in StrataGraft (Table 10). At Day 28, 42.9% (27/63) of patients had reactivity to alleles found in StrataGraft, and this decreased to 26.3% (15/57) of patients by Month 3 (Table 10). In comparison, at Day 28, 57.1% (36/63) of patients demonstrated reactivity to alleles not found in StrataGraft, and this increased to 73.7% (42/57) at Month 3 (Table 10). At Month 3, no patients (n=57) tested positive for residual allogeneic DNA at their StrataGraft treatment site. Anti-BSA antibody levels increased in 16.9% (12/71) of patients from baseline (Day 0) to Month 3.

Discussion: Autografting is the standard of care for severe burns, including DPT burns, and requires surgical care.^{19,20} However, donor sites that are created during the procedure are painful, may result in sequelae that require additional treatment, and contribute to increased patient morbidity.¹⁹ As a result, there is an unmet need for clinical approaches and treatment options that can provide predictable and efficient burn wound closure in the absence or with reduction of autografting.

This phase 3, open-label, controlled, randomized, multicenter study evaluated the efficacy and safety of StrataGraft, a bilayered bioengineered regenerative skin construct that recapitulates the structural and biological properties of the dermis and epidermis, for the treatment of DPT thermal burns requiring surgical excision and autografting. Both coprimary endpoints were achieved. StrataGraft treatment resulted in a 96% reduction relative to autograft control, a significant difference in the mean percentage of treatment site autografted, and 83% of patients, a significant proportion, achieved durable wound closure of the StrataGraft treatment site by Month 3 in the absence of autografting. Our data demonstrate that both StrataGraft and control autograft treatment achieved the protocol-defined time for durable wound closure. However, for StrataGraft-treated wounds, this was achieved in nearly all patients without the burden of creating a donor-site wound. Notably, missing data for coprimary endpoint data were imputed as a “failure” for both treatment arms, thereby lowering the percentage of patients who achieved wound closure with StrataGraft and autograft. Moreover, a post hoc analysis of wound closure using the definition applied to the STRATA2011 study¹⁶ (i.e., 95% re-epithelialization without drainage), demonstrates that 87% (95% CI: 80%-95%) of patients in each treatment group achieved durable wound closure at Month 3.

In terms of the safety profile, pruritus was the most common TEAE observed. Further, TEAEs observed in this study are consistent with those commonly seen in other patients who have burns treated with standard approaches. By investigator assessment, 3 patients had hypertrophic scar formation related to StrataGraft treatment. However, the incidence and/or prevalence of hypertrophic scars may change as scars mature and the majority of patients represented in the dataset were less than 1-year postgrafting. Infection at a single StrataGraft treatment site was reported and that patient had their StrataGraft site autografted after treatment of the infection.

Cosmesis data at Month 3 showed a significantly favorable difference in mean total POSAS scores between StrataGraft and autograft donor sites (P<0.0001). Although most DPT wounds are expected to heal within 3 months without autograft, such wounds are often associated with elevated POSAS scores (indicative of poorer cosmesis). At the time of data cutoff in this study, cosmesis data at treatment sites at Month 12 (secondary endpoint), was available for only 22 patients and the results were comparable between the StrataGraft and autograft treatment sites.

The proportion of patients with reactivity to alleles found in StrataGraft increased between baseline and Day 28 (4.3 to 42.9%), and subsequently decreased between Day 28 and Month 3 (42.9 to 26.3%). The clinical relevance of these findings remains uncertain; however, no patients in this study showed clinical signs of StrataGraft tissue rejection. In addition, patients received other allogeneic products during the course of standard treatment for severe burns that may have impacted the PRA assay results. This may likely account for the increasing percentage of patients over time with reactivity to alleles not found in StrataGraft. Further, molecular analysis of patient biopsies at the StrataGraft treatment site demonstrated that DNA from cells used to produce StrataGraft was not detectable at Month 3 in all patients evaluated. Overall, <20% of patients demonstrated an increase in the presence of anti-BSA antibodies from baseline to Month 3.

A significant difference in donor-site pain intensity through Day 14 was observed between StrataGraft and autograft donor sites (P<0.0001). Less pain at the StrataGraft donor site was expected, given that the prospective donor sites for StrataGraft (healthy skin) were harvested only if needed. In this study, only 3 patients subsequently required autograft at their StrataGraft treatment sites. The mean percentage area of control treatment sites that received autograft was 102.1%, reflecting the need for additional autograft (i.e., regrafting) at the autograft treatment site.

In summary, this phase 3 study demonstrates that StrataGraft treatment results in durable wound closure of DPT thermal burns, while also reducing the harvest and use of autograft. Both donor-site pain and donor-site cosmesis were favorable outcomes of significantly reduced use of autograft in StrataGraft-treated patients. Taken together, StrataGraft may offer an alternative and attractive treatment option for DPT burns, with the potential to reduce or eliminate the need for autografting and associated wound-healing sequelae.

Lastly, StrataGraft represents a potential treatment for older adult patients who have a higher risk of delayed or impaired wound healing, as well as other patients who have a higher risk of delayed or impaired wound healing due to comorbidities.

### REFERENCES

1. Heimbach, D. M., G. D. Warden, A. Luterman, et al., Multicenter postapproval clinical trial of Integra dermal regeneration template for burn treatment. J Burn Care Rehabil 2003; 24: 42-8.
2. Shimizu, R. and K. Kishi, Skin graft. Plast Surg Int 2012; 2012: 563493.
3. Sinha, S., A. J. Schreiner, J. Biernaskie, et al., Treating pain on skin graft donor sites: Review and clinical recommendations. J Trauma Acute Care Surg 2017; 83: 954-964.
4. Guo, S. and L. A. Dipietro, Factors affecting wound healing. J Dent Res 2010; 89: 219-29.
5. Seidenari, S., G. Giusti, L. Bertoni, et al., Thickness and echogenicity of the skin in children as assessed by 20-MHz ultrasound. Dermatology 2000; 201: 218-22.
6. Greenhalgh, D. G., Management of the skin and soft tissue in the geriatric surgical patient. Surg Clin North Am 2015; 95: 103-14.
7. Janzekovic, Z., Early surgical treatment of the burned surface. Panminerva Med 1972; 14: 228-32.
8. Janzekovic, Z., Past and present management burns. Magy Traumatol Orthop Helyreallito Seb 1975; 18: 260-4.
9. Janzekovic, Z., The burn wound from the surgical point of view. J Trauma 1975; 15: 42-62.
10. Stone, R., S. Natesan, C. J. Kowalczewski, et al., Advancements in Regenerative Strategies Through the Continuum of Burn Care. Front Pharmacol 2018; 9: 672.
11. Wainwright, D. J. and S. B. Bury, Acellular dermal matrix in the management of the burn patient. Aesthet Surg J 2011; 31: 13S-23S.
12. Carsin, H., P. Ainaud, H. Le Bever, et al., Cultured epithelial autografts in extensive burn coverage of severely traumatized patients: a five year single-center experience with 30 patients. Burns 2000; 26: 379-87.
13. Vig, K., A. Chaudhari, S. Tripathi, et al., Advances in Skin Regeneration Using Tissue Engineering. Int J Mol Sci 2017; 18.
14. Rennert, R. C., M. Rodrigues, V. W. Wong, et al., Biological therapies for the treatment of cutaneous wounds: phase III and launched therapies. Expert Opin Biol Ther 2013; 13: 1523-41.
15. Allen-Hoffmann, B. L., S. J. Schlosser, C. A. Ivarie, et al., Normal growth and differentiation in a spontaneously immortalized near-diploid human keratinocyte cell line, NIKS. J Invest Dermatol 2000; 114: 444-55.
16. Holmes, J. H., M. J. Schurr, B. T. King, et al., An open-label, prospective, randomized, controlled, multicenter, phase 1b study of StrataGraft skin tissue versus autografting in patients with deep partial-thickness thermal burns. Burns 2019; 45: 1749-58.
17. Wong, D. L. and C. M. Baker, Pain in children: comparison of assessment scales. Pediatr Nurs 1988; 14: 9-17.
18. Draaijers, L. J., F. R. Tempelman, Y. A. Botman, et al., The patient and observer scar assessment scale: a reliable and feasible tool for scar evaluation. Plast Reconstr Surg 2004; 113: 1960-5; discussion 1966-7.
19. Rowan, M. P., L. C. Cancio, E. A. Elster, et al., Burn wound healing and treatment: review and advancements. Crit Care 2015; 19: 243.
20. Wurzer, P., H. Keil, L. K. Branski, et al., The use of skin substitutes and burn care—a survey. J Surg Res 2016; 201: 293-8.

TABLE 6
Patient demographic and baseline characteristics, ITT population

| | All Patients | |
|---|---|---|
| | (N = 71) | |
| Age^{a} (years), median (min, max) | 45 | (19, 79) |
| Age category (years), n (%) | | |
| <65 | 63 | (88.7) |
| ≥65 | 8 | (11.3) |
| Sex, n (%) | | |
| Male | 55 | (77.5) |
| Female | 16 | (22.5) |
| Ethnicity, n (%) | | |
| Hispanic or Latino | 10 | (14.1) |
| Not Hispanic or Latino | 61 | (85.9) |
| Race, n (%) | | |
| White | 55 | (77.5) |
| Black or African American | 14 | (19.7) |
| Asian | 1 | (1.4) |
| Other | 1 | (1.4) |
| BMI (kg/m²), median (min, max) | 29.2 | (20.2, 52.3) |
| Baux score,^{b} median (min, max) | 57.0 | (23.0, 91.75) |
| Modified Baux score,^{c} median (min, max) | 57.0 | (23.0, 108.75) |
| Total body surface area burned (% TBSA),^{d} median (min, max) | 9.0 | (3.0, 36.5) |
| % TBSA, mean (SD) | 12.0 | (8.4) |
| StrataGraft treatment area (cm²) | | |
| Mean (SD) | 239.8 | (202.2) |
| Median (min, max) | 162.0 | (12, 960) |
| Autograft treatment area (cm²) | | |
| Mean (SD) | 219.8 | (233.2) |
| Median (min, max) | 130.0 | (20, 1330) |
| Size of StrataGraft treatment area (cm²), n (%) | | |
| <250 | 47 | (66.2) |
| 250-500 | 17 | (23.9) |
| ≥500 | 7 | (9.9) |

^{a}Age is calculated as the number of years (the patient's informed consent date − date of birth)/365.25, rounded down to the nearest integer.
^{b}Baux score = Age + total body surface burned. TBSA includes only second- and third- degree burns.
^{c}Modified Baux score = age + total body surface burned + [17 × (inhalation injury, 1 = yes, 0 = no). TBSA includes only second- and third-degree burns.
^{d}Represents the sum of second- and third degree burns.
BMI, body mass index; ITT, intention-to-treat.

TABLE 7
POSAS observer total scores at Month 3, mITT population

| Assessment | Treatment Site | n | Mean (SD) | P Value^{a} |
|---|---|---|---|---|
| Donor-site POSAS total | StrataGraft | 61 | 6.3 (1.71) | |
| score^{b} at Month 3 | Autograft | 61 | 16.3 (7.71) | |
| | Difference | | 10.0 (7.92) | <.0001 |

^{a}P value from one-sided, paired t-test on the difference (autograft − StrataGraft).
^{b}Total score is the sum of the scores for vascularization, pigmentation, thickness, relief, pliability, and surface area.
mITT, modified intention-to-treat; POSAS, Patient and Observer Scar Assessment Scale; SD, standard deviation.

TABLE 8
Summary of treatment-emergent adverse events
reported in ≥5% of patients, safety population

| | Related to | Not related to | |
|---|---|---|---|
| System Organ Class and | StrataGraft | StrataGraft | |
| Preferred Term | N (%) | N (%) | |
| Skin and subcutaneous disorders | | | |
| Pruritus | 11 (15.5) | 14 | (19.7) |
| Hypertrophic scar | 3 (4.2) | 8 | (11.3) |
| Blister | 4 (5.6) | 6 | (8.5) |
| Rash | 0 | 4 | (5.6) |
| Gastrointestinal disorders | | | |
| Constipation | 0 | 9 | (12.7) |
| Nausea | 0 | 5 | (7.0) |
| General disorders and site conditions | | | |
| Pain | 1 (1.4) | 7 | (9.9) |
| Pyrexia | 0 | 5 | (7.0) |
| Injury, poisoning, and procedural complications | | | |
| Donor-site complication | 0 | 5 | (7.0) |
| Musculoskeletal disorders | | | |
| Muscle spasms | 0 | 6 | (8.5) |
| Vascular disorders | | | |
| Hypertension | 0 | 6 | (8.5) |
| Psychiatric disorders | | | |
| Insomnia | 0 | 5 | (7.0) |
| Nervous system disorders | | | |
| Neuralgia | 1 (1.4) | 6 | (8.5) |
| Blood and lymphatic system disorders | | | |
| Anemia | 0 | 4 | (5.6) |

TABLE 9
Serious treatment-emergent adverse events, safety population

| | All Patients | |
|---|---|---|
| SOC | (N = 71) | |
| Preferred Term | Related to StrataGraft | Not Related to StrataGraft |
| Total number of SAEs | 0 | 21 |
| Number of subjects with ≥1 SAE | 0 | 10 (14.1) |
| General disorders and administration- | | |
| site conditions | | |
| | 0 | 3 (4.2) |
| Concomitant disease progression | 0 | 1 (1.4) |
| Death | 0 | 1 (1.4) |
| Malaise | 0 | 1 (1.4) |
| Pyrexia | 0 | 1 (1.4) |
| Cardiac disorders | | |
| | 0 | 2 (2.8) |
| Acute left ventricular failure | 0 | 1 (1.4) |
| Acute myocardial infarction | 0 | 1 (1.4) |
| Atrial fibrillation | 0 | 1 (1.4) |
| Bundle branch block left | 0 | 1 (1.4) |
| Cardiac arrest | 0 | 1 (1.4) |
| Infections and infestations | | |
| | 0 | 2 (2.8) |
| Enterobacter bacteremia | 0 | 1 (1.4) |
| Pneumonia bacterial | 0 | 1 (1.4) |
| Pseudomonal bacteremia | 0 | 1 (1.4) |
| Sepsis | 0 | 1 (1.4) |
| Vascular disorders | | |
| | 0 | 2 (2.8) |
| Deep vein thrombosis | 0 | 1 (1.4) |
| Migraine | 0 | 1 (1.4) |

Data are presented as n (%).
Adverse events were coded by MedRA Version 19.1.
The number of patients is summarized.
Percentages are based on the number of patients in each treatment group.
The total number of treatment-emergent adverse events (TEAEs) includes all TEAEs that occurred during or after the date of StrataGraft or autograft placement.
Patients may have >1 TEAE per SOC and preferred term within relationship.
If so, TEAEs were counted once within relationship prioritizing the related TEAE for this summary.
MedRA, Medical Dictionary for Medical Activities; SAE, serious adverse event; SOC, system organ class.

TABLE 10
Panel reactive antibody test-positive patients

| | Patients with reactivity to | | Patients with reactivity to alleles not | |
|---|---|---|---|---|
| Time of PRA | alleles found in StrataGraft | | found in StrataGraft | |
| assessment | N (%) | | N (%) | |
| Baseline (n = 70) | 3 | (4.3) | 67 | (95.7) |
| Day 28 (n = 63) | 27 | (42.9) | 36 | (57.1) |
| Month 3 (n = 57) | 15 | (26.3) | 42 | (73.7) |

PRA, panel reactive antibody

### Example 4

This example describes the successful use of StrataGraft in subjects at risk of impaired or delayed wound healing.

TABLE 11
Subjects Treated with StrataGraft Skin Tissue
as Part of an FDA Expanded-Access Program

| Case # | Sex, Age | Injury | Reported SAEs | Outcome (Wound closure) |
|---|---|---|---|---|
| 1 | Male, 29 yrs. | Explosion, | Fungal sepsis, | Died 3 weeks after surgery |
| | | Thermal FT + | Multisystem | for autograft and StrataGraft |
| | | DPT burn (84% | organ failure | application due to causes |
| | | TBSA) | | unrelated to StrataGraft |
| | | | | treatment - StrataGraft used |
| | | | | as part of a sandwich graft |
| | | | | on FT burn |
| 2 | Male, 4 yrs. | Flame DPT + FT | None | Applied total of 12 tissues |
| | | burn (79% TBSA) | | on Day 0 and Day 6. As of |
| | | | | Month 6 + 10 days, patient |
| | | | | is home and doing well. |
| | | | | Wounds are all healed. |
| 3 | Male, 15 mos. | Scald DPT + FT | None | 2 StrataGraft tissues |
| | | burns (29.5% | | applied to scalp and face |
| | | TBSA) | | DPT burns. |
| 4 | Male, 9 wks. | Contact DPT burn | None | <1 StrataGraft tissue |
| | | (2% TBSA) | | applied; Healed at 2 weeks |
| | | | | and home. |
| 5 | Male, 33 yrs. | Flame DPT + FT | None | 21 StrataGraft tissues |
| | | burn (40% TBSA) | | applied (sandwich graft of |
| | | | | FT area on right leg, alone |
| | | | | on DPT burn of forearm, |
| | | | | and on donor site[s]) 27 Nov. |
| | | | | 2019: he is at home and |
| | | | | doing well. |

SAE = serious adverse event; DPT=deep partial-thickness; FDA=Food and Drug Administration; FT=full-thickness; TBSA=total body surface area.
Note:
All subjects were treated with 100 cm² rectangular format StrataGraft skin tissues that had been stored under ultracold conditions.

1. A method for closing an acute wound in a subject, the method comprising applying a skin substitute over an acute wound and allowing the wound to heal, wherein the skin substitute is an organotypic human skin equivalent comprising NIKS.

2. The method of claim 1, wherein the acute wound contains intact dermal elements.

3. The method of claim 1, wherein wound closure occurs without the application of autologous tissue or without the application of any autologous cells.

4. The method of claim 1, wherein the closed wound has improved vascularity, improved pigmentation, decreased thickness, decreased pain, increased pliability, increased surface area, decreased stiffness, decreased itching, improved color, or any combination thereof, as assessed by an observer or by the subject, as compared to an autograft or another skin substitute.

5. A method for improving an outcome of skin grafting in a subject, the method comprising applying a skin substitute over an acute wound, wherein the skin substitute is an organotypic human skin equivalent comprising NIKS, and wherein the outcome is reduced autografting, improved vascularity, improved pigmentation, decreased thickness, decreased pain, increased pliability, increased surface area, decreased stiffness, decreased itching, improved color, decreased infection rate or any combination thereof, as assessed by an observer or by the subject, as compared to an autograft.

6. The method of claim 5, wherein the acute wound contains intact dermal elements.

7. The method of claim 5, wherein the improved outcome occurs without the application of autologous tissue or without the application of any autologous cells.

8. The method of claim 5, wherein the improvement is statistically significant.

9. The method of claim 5, wherein the acute wound is a partial thickness wound.

10. The method of claim 5, wherein the acute wound is a complex wound with intact dermal elements.

11. The method of claim 1, wherein the acute wound is a full thickness wound.

12. The method of claim 1, wherein the acute wound is a burn wound.

13. The method of claim 12, wherein the acute wound is an electrical burn wound, a chemical burn wound, or a thermal burn wound.

14. The method of claim 1, wherein the acute wound is a surgical wound.

15. The method of claim 1, wherein the subject has a total wound area covering up to about 85% total body surface area (TBSA), the total wound area comprising the area of the acute wound.

16. The method of claim 15, wherein the method further comprises applying the skin substitute to a plurality of acute wounds.

17. The method of claim 16, wherein the skin substitute is applied to about 1% to about 100% of the total wound area.

18. (canceled)

19. (canceled)

20. (canceled)

21. (canceled)

22. The method of claim 1, wherein the organotypic skin equivalent comprises a dermal equivalent, the dermal equivalent comprising gelled collagen containing normal human dermal fibroblasts.

23. The method of claim 22, wherein the collagen present in the dermal equivalent comprises type I murine collagen.

24. The method of claim 22, wherein the only collagen in the dermal equivalent is produced by cells of the skin substitute.

25. The method of claim 1, wherein the wound is clinically indicated for excision and grafting.

26. The method of claim 1, wherein the subject is not a candidate for an autograft.

27. The method of claim 1, wherein the subject is less than 18 years of age or greater than 65 years of age.

28. (canceled)

29. (canceled)

30. (canceled)

31. (canceled)

32. The method of claim 1, wherein the skin substitute is applied to a surface area of less than 200 cm².

33. The method of claim 1, wherein the skin substitute is applied to a surface area of 200 cm² to about 15,000 cm².

34. (canceled)

35. (canceled)

36. (canceled)

37. (canceled)

38. The method of claim 1, wherein wound closure occurs by about 2 weeks to about 12 weeks.

39. (canceled)

40. (canceled)

41. (canceled)

42. The method of claim 1, wherein wound closure is defined as about 90% re-epithelialization or greater.

43. (canceled)

44. (canceled)

45. (canceled)

46. The method of claim 1, wherein wound closure is defined as durable wound closure.
